(19) United States
(12) Patent Application Publication
(10) Pub. No.: US 2026/0226544 A1
(43) Pub. Date: Aug. 6, 2026

(54) METHOD FOR CHARACTERIZING THE ONSET, DURATION, AND RESOLUTION OF THE BIOLOGICAL EFFECTS OF A THERAPEUTIC PRODUCT

(71) Applicant: Bios-Therapy, Physiological Systems for Health S.p.A., Sansepolcro (IT)

(72) Inventors: Valentino MERCATI, Sansepolcro (IT); Jacopo LUCCI, Sansepolcro (IT)

(73) Assignee: Bios-Therapy, Physiological Systems for Health S.p.A., Sansepolcro (IT)

(21) Appl. No.: 19/261,022

(22) Filed: Jul. 7, 2025

(30) Foreign Application Priority Data
Feb. 3, 2025 (EP) 25155583.5
Jul. 4, 2025 (WO) PCT/IB2025/056792

(51) Int. Cl. C12Q 1/6876

(52) U.S. Cl. CPC C12Q 1/6876; C12Q 2600/136; C12Q 2600/158

(57) ABSTRACT

The present invention provides an innovative analytical strategy specifically designed to overcome the inherent limitations of the traditional ADME (Absorption, Distribution, Metabolism, Excretion) approach in the pharmacokinetic and pharmacodynamic characterization of natural matrices-based therapeutic or beneficial product, such as a product comprising or consisting of multicomponent botanical extracts.

The invention provides a fundamentally different approach that is not based on the molecular profile (i.e., an approach independent to the standardisation of each and every “marker”) of the administered therapeutic or beneficial product. The novel approach proposed herein, captures the integrated, functional response of the organism to the administration of a therapeutic or beneficial product, i.e., relies on a methodology that characterizes the pharmacokinetic and pharmacodynamic behaviour of a therapeutic or beneficial product through functional biological response, independent of its chemical composition. This approach, although generally applicable also to conventional drugs and beneficial products, is particularly suitable for multicomponent products such as products comprising natural matrices, e.g., plant natural matrices such as plant extracts, parts, fractions or the like.

### TECHNICAL FIELD OF THE INVENTION

The present invention relates to an improved method for functionally characterising the onset, duration, and resolution of the biological effects of a medicinal product following its administration to a subject. The method characterizes therapeutic or beneficial products and is particularly suitable for natural matrices-based products, i.e., compositions of matter consisting solely of natural matter and having a physiological mode of action.

### PREMISE

The invention relates to products wherein native matrices, appropriately processed through specific processes and methods, and assembled, create final products intended for therapeutic or beneficial purposes, for restoring or adjuvating the organism in restoring healthy physiological states. Every phase of the production process of such products is under the aegis of the One Health principle (which is a principle that recognizes the interconnectedness of human health, animal health, and environmental health), therefore, the use of artificial forces or substances is not permitted.

Indeed, the present invention, is particularly addressed products comprising or consisting of one or more natural matrices, must maintain the natural intelligence i.e. the imprint of the domain of the living to which each constituent of the product belongs, thereby maintaining a network capable of interconnecting and recognizing itself with other networks, whether natural or artificial, that is, originally natural networks that have acquired a degree of artificiality due to interaction with artificial components. This interconnection is deemed to be fundamental to rebalance any disturbances in the network of events that are active in each interacting biological system. Every matrix identified will present bio-physical specifications such that it will represent an invention on its own.

Each network of each native natural matrix comprised in the product, which will contribute to forming the network of the final matrix of the product of the invention, can be defined as a UVCB substance (i.e. Substances of Unknown or Variable composition, Complex reaction products, or Biological materials) according to the REACH (Registration, Evaluation, Authorisation, and Restriction of Chemicals) definition since, being a product processed respecting its self-assembly peculiarities, it cannot be determined nor validated on the basis of small molecule chemistry protocols.

Each network will be characterized by the establishment of connections within the matrices in the final product and within the physiological actions exerted by the product on the receiving organism. The production validation of the product can be carried out and confirmed using probabilistic models based on the link between the conservation of a physiological activity profile and descriptors of the matrix per se generated using multiple bio-physical analytical systems, including spectroscopy (NIR and other techniques), mass spectrometry, and paper or X-ray crystallography (fractal measurements).

Natural matrices, such as those forming the basis of the products of interest in present invention, contain a multitude of constituents whose interactions, both at the structural and functional levels, produce a dynamic and holistic biological response. Some components may modulate the bioavailability of others, while synergistic or sequential actions on multiple molecular pathways generate pharmacodynamic effects that cannot be linked to the plasma concentration of a single active principle. Moreover, it is frequently observed that biological effects persist beyond the detectability of any individual constituent in the bloodstream, reflecting genuine phenomena inherent to the complex network of actions triggered by natural matrices.

Therefore, the conventional pharmacokinetic parameters such as Cmax, Tmax, AUC, and systemic clearance are not fully representative or adequate for characterizing these types of products. Instead, alternative analytical strategies, focusing on the organism-level physiological response and inter-network communication, are required to validate the effects of the invention.

This paradigm shift aligns perfectly with the objectives of the Bios Physiological Health model proposed herein, emphasizing the rebalancing of the psycho-neuro-endocrine-immunological system through endogenous activation mechanisms rather than relying on isolated exogenous substances. Although useful, the conventional molecular chemical definitions of individual substances contained in matter cannot be used for validating this kind of products as they are not representative of the overall effectiveness and quality thereof.

The selection of matrices intended for administration must be validated according to the updated and specific current taxonomic criteria for the animal, plant, and mineral kingdoms. In case of use in combination with natural physical phenomena, it will be necessary to validate the relationship between action and effectiveness on a case-by-case basis, considering sound effects (music or other forms) and those in the field of wave-particles, including those of a quantum nature.

In the current state of the art, it will not always be possible to outline a fully described mechanism of action; however, it will be possible to validate the action and reaction in the interconnection of the respective networks, already validated at a biophysical level.

The invention aims to select and provide new entities or products, as well as systems capable of rebalancing, activating, or limiting physiological functions in specific metabolic states of living organisms, that are always in continuous transformation.

The preparations conceived in this way can rebalance the psycho-neuro-endocrine-immunological system, considered as a single system that governs and manages all the other systems.

The invention contributes to a new state of the art that goes beyond the alchemical technologies in the medical field, the beginning of which can be traced back to the early years of the sixteenth century, bringing products and processes back to the conceptual One Health objective already mentioned. The invention proposes a new declination of artificial technologies and of those that naturally self-assemble matter, recognizing existing rules or finding new ones in order to guarantee the constitution of entities that can be validated, mainly based on the concept of validating of their effect and activity on other organisms. The latter, being living beings in continuous transformation, require an evaluation of their physiological state within defined intervals, which is a concept that today falls within the personalized medicine. The present invention fits into the concept of scientificity, understood as the set of knowledge that can demonstrably validate the effects of theoretical modes of action.

Since the activities of the invention herein disclosed are not currently covered by the state of the art, it will be necessary to consider the entire product cycle, from the end user to the social context concerned, under the concept of One Health.

The operational paradigm within which the invention was prepared has been herein denominated as “Bios Physiological Health”.

This paradigm aims to introduce into the field of medical art an innovative approach for the treatment and self-management of health using natural matrices, alone or in combination, in order to rebalance the normal physiological states of various living entities, including humans, through endogenous physiological actions triggered by the product. It is a matter of identifying, selecting, and assembling natural entities which possess emerging properties, validable through the final product's physiological mode of an action and other methods evolved in recent decades.

A reading of the context according to both technical-scientific and humanistic canons, integrated in their transversality, constitutes the foundation of the proposed invention. Although some of the properties of each matrix part of the product may already be known the emerging properties of the new composition are unexpected.

Of relevance is the role of determining the genetic and epigenetic aspects determining the network representing natural matrices and their description at the level of their specific isotopic abundance.

In order to fulfil the Bios Physiological Health paradigm, each phase of processing, from the selection of the reproductive material to the agricultural and industrial phases, up to the methods of use, must preserve as much as possible the integrity of the native programming heritage, inserted into the natural intelligence of each entity of creation, at least as far as known in our terrestrial dimension. It will be essential to validate matrices coming from epigenetic realities similar to the reference one, recognized as a Reference Standard for its specific emerging properties on the metabolism of other living beings, including humans. By way of example, one of the factors that negatively influences epigenetic differentiations is represented by different soil conditions, together with circadian, monthly, and annual variations. To preserve the properties of the natural system, which are the only ones that can claim physiological interconnection with the whole of creation, it is not possible to use substances derived from alchemical processes, such as distillation, other processes of synthesis or hemi synthesis, or products derived from genetic modifications or genetically modified organisms. A new interpretation of the mysteries of natural programming, responsible for the vital evolution of organic and inorganic matter, is needed. The consolidation of scientific evolution in recent decades allows us to reposition the understanding of the genesis of a progress based on reductionist determinism, founded on the development of alchemical processes starting from the beginning of the 16th century, which in medicine, with Paracelsus, marked the beginning of the current evolutionary process, known as the Anthropocene.

The term Anthropocene describes the current phase of human evolution and can be dated back to different eras. If considered in the context of this invention, the key date can only be 1492, which represents the end of the humanistic/neo platonic period of the early Renaissance. This period was represented politically by Cosimo the Elder and Lorenzo de' Medici, with artists and scientists such as Piero della Francesca, Luca Pacioli, Leonardo and Durer. In the 16th century, alchemical research seen as a human possibility of dominion over nature, has evolved until today under the aegis of artificial intelligence as opposed to the natural one, inspired from the biblical thought according to which “man will dominate over all creation”, with the aim of improving divine creation.

1492 is a symbolic date: in that year Lorenzo de′ Medici and Piero della Francesca died, while Columbus discovered America. The human species abandoned the fifteenth-century Neoplatonic path to follow the Judeo-Catholic one, where the alchemical practices of Paracelsus applied to medicine marked the transition to the Renaissance mannerism of the 1500s, which, up to the present day, has led us towards a full-blown and irreversible sixth extinction.

The present invention, with the demonstration of feasibility of the resulting industrial discoveries in the medical field, but in principle adaptable to any production field, intends to address the change in evolutionary paradigms. We often talk about defending biodiversity without ever addressing the real problem, guiltily obscured, of the billions of tons of exogenous and non-biodegradable artificial substances released into the planetary system, with the certainty of irreversibly poisoning the sources of life, while the “carpe diem” approach prevails over the survival instinct of the species.

The invention is presented primarily in the patent context, with the hope of opening new areas of research that explore and share natural intelligence, rather than artificial intelligence, which, can do very little to stop or slow down the sixth extinction, or to lay the foundations of an alternative progress to the current one. The inventor Valentino Mercati, together with his collaborator Jacopo Lucci, has undertaken the path of researching in nature itself what can be useful to the living systems, and has developed some knowledge in the agricultural and industrial production system for over 40 years, presenting numerous patent applications following this operational strategy. The patents filed in the past relating to the present inventive process are essentially based on instrumental and diagnostic readings based on principles related to chemistry for the connection of physiological actions with the emerging properties of natural matrices and the innate defences of each individual living being with which they interconnect.

The analyses that followed and inspired the approach herein disclosed were unthinkable just a few decades ago, due to the technological impossibility of reading the genetic and epigenetic information written in the cells of every living organism, and the role of atomic isotopic differentiations in the molecular self-assembly and interconnections of every single unity/individuality with the “universe”. The conceptual difficulty in moving from the reassuring management parameters of the artificiality of molecules—at least partly purified and linked by powerful thermodynamic forces that allow strong bonds such as covalent ones, which acts on reduced molecular scopes of other organisms—to natural matrices, mysterious by definition and, still viewed today as part therapeutically unreliable, is extremely high.

If a new inventive interpretation is needed for a new medical state of the art after five centuries of alchemical reductionism, this interpretation must connect the most distant concepts and processes in a single application field. This is due, as already expressed, to a philosophical legacy that questions the human condition: the human species was created like all the others by the original vital intelligence with the purpose of life itself, as far as we can assume, to dominate on creation, or has it been experimentally endowed with different faculties from other organisms, already favourably inserted into creation, to constitute a new ecological niche at the service of the universe?

The answer to this dilemma does not arise for the current invention: humanity will have to return to the Neoplatonic thought of the early Renaissance, and the experimental duality of the human species must emancipate itself from the mindset of dominance in order to share its unique faculties within the universe with all of creation. Humanity will need to reconsider the warning of Leonardo da Vinci: “Man can only create his own offspring . . . ” and reflect on the melancholic thoughts of wise figures like Piero della Francesca, Luca Pacioli, and Durer, regarding the impossibility of understanding and representing the beauty of creation and deciphering its mysteries.

The era has arrived for the acquisition of new research centres in molecular and cellular biology, with an indispensable focus on bioinformatics and the new physical sciences. Today, the inventor can base research strategies and socioeconomic applications in new therapeutic fields, particularly those that are complex and/or chronic-degenerative, where the restoration of metabolic balance for organisms either naturally or artificially disturbed will become an integral part of a future that is already present.

The present invention represents a new vision of the medical art, which reconsiders scientific evolution from a perspective different from reductionist determinism. This alternative progress, in conflict with universal or planetary rules, will have to rely not so much on artificial intelligence and technological advancement, but on the evolution of the laws that regulate our universe and life itself. The transition from the artificial treatment of singled out symptoms, even if seen from a systemic perspective with modern techniques of systems biology, to a holistic approach that embraces the whole, represents the basis of the present progress.

In this context, the invention addresses the urgent need to provide validation, safety, and functional evaluation of the organism's global biological response to the administration of therapeutic or beneficial products based exclusively on natural matrices that operate through mechanisms that respect the intrinsic complexity of living systems.

Rather than depending on the conventional metrics of pharmacokinetics designed for simple molecules, the invention validates its effectiveness through the conservation and modulation of physiological network activities across psycho-neuro-endocrine-immunological systems.

The Bios Physiological Health paradigm proposed herein establishes a new scientific canon: one that measures health intervention success not by isolated chemical concentrations, but by the organism's restored ability to maintain coherence within its internal networks and to re-establish dynamic balance with its environment.

Thus, the invention opens new research frontiers focused on the deep understanding of natural intelligence and its synergistic interactions with the living, offering an authentic alternative to the current models, and aiming to contribute to the survival and regeneration of life forms in an era of profound ecological and biological disruption.

### BACKGROUND OF THE INVENTION

In the pharmaceutical sciences, the characterization of a compound's pharmacokinetic profile traditionally follows the ADME paradigm (Absorption, Distribution, Metabolism, and Excretion). This analytical framework has been instrumental in the development and regulatory approval of numerous pharmacological agents, as it enables the definition of key parameters such as half-life, peak plasma concentration (Cmax), time to peak concentration (Tmax), area under the curve (AUC), and systemic clearance.

Such metrics are derived from direct quantification of the active ingredient in plasma or tissues over time, and they form the backbone of most pharmacokinetic and pharmacodynamic models in modern drug development. However, this approach is inherently tailored to chemically homogeneous substances-namely, small molecules with a single, well-defined active principle whose biological activity is closely correlated with measurable systemic exposure. In these contexts, the underlying assumption is that the therapeutic effect is tightly linked, both temporally and quantitatively, to the detectable presence of the drug or its metabolites in the bloodstream and target tissues.

This assumption becomes problematic when extended to multicomponent botanical extracts, or plant natural matrices, which are structurally and functionally heterogeneous by nature. Such extracts typically contain dozens to hundreds of distinct chemical constituents. These compounds may act synergistically, antagonistically, or sequentially across multiple biological targets and pathways.

These interactions may occur at a structural level where different molecules modulate each other's absorption, bioavailability, or metabolic fate, potentially through complex formation, micelle generation, or competition for transporters, and at a functional level where the combined effect results from the activation or inhibition of multiple cellular or molecular pathways, creating a pharmacodynamic footprint that is not attributable to any single component.

Furthermore, the pharmacokinetic behaviour of such extracts is intrinsically difficult to dissect. Some components may be rapidly metabolized and eliminated, others may accumulate in tissues, and still others may exert biological effects indirectly by modulating endogenous signalling cascades or by generating active metabolites. Attempting to define the “effective” concentration of a plant natural matrix based on the plasma levels of a few arbitrarily selected marker compounds is therefore an oversimplification that ignores the holistic and interactive nature of these products. Moreover, botanical matrices act not on isolated molecular targets but on the whole pathological state, aiming to restore the dynamic physiological balance of the organism through endogenous regulatory mechanisms rather than through a purely pharmacological suppression or stimulation. In this perspective, following the ADME parameters of individual compounds within a natural matrix, such as a plant natural matrix, is inherently inadequate, as absorption, distribution, metabolism, and excretion of isolated molecules do not represent the full systemic action and physiological rebalancing triggered by the natural matrix as a whole.

This conceptual and methodological limitation has concrete implications: when traditional ADME strategies are applied to complex botanical products, they often fail to fully account for the observed biological activity or its persistence over time. In some cases, pharmacodynamic effects are detected even when no trace of the presumed active constituents is measurable in plasma or tissues. Such findings are frequently dismissed as “anomalous” or attributed to analytical shortcomings, yet they may reflect genuine biological phenomena intrinsic to the multi-layered action of complex natural products.

Critically, this disconnect between detectable pharmacokinetic exposure and observed biological effect is not unique to natural matrices-based therapeutic or beneficial products. Even conventional drugs—whose pharmacokinetic behaviour can be precisely characterized—exhibit well-documented cases of prolonged or persistent effects despite the absence of a detectable drug in systemic circulation. These phenomena challenge the simplistic correlation between drug presence and pharmacological effect and underscore the existence of mechanisms capable of sustaining biological activity well beyond drug clearance.

Several conventional drugs exhibit pharmacodynamic effects that persist beyond their measurable presence in plasma, clearly showing that traditional ADME evaluation provides only a partial understanding of their biological actions. A well-known example is the post-antibiotic effect (PAE), where bacterial growth remains suppressed even after antibiotic concentrations fall below detectable levels. This phenomenon, documented for multiple classes of antimicrobials such as aminoglycosides and fluoroquinolones, is attributed to lingering sub-lethal bacterial damage and host immune system involvement, and has practical implications for dosing strategies, allowing for extended intervals without loss of efficacy (Craig W A. Pharmacokinetic/pharmacodynamic parameters: rationale for antibacterial dosing of mice and men. Clin Infect Dis. 1998 January; 26(1):1-10; quiz 11-2. doi: 10.1086/516284.)

Persistent effects are also observed with drugs that irreversibly bind to their biological targets. Aspirin, for instance, permanently acetylates platelet cyclooxygenase-1 (COX-1), leading to inhibition of thromboxane A₂ synthesis for the lifespan of the platelet, even though aspirin itself is cleared from the bloodstream within hours. This long-lasting antiplatelet effect is central to its cardioprotective use and underlies clinical recommendations such as stopping aspirin therapy a week before surgery (Vane J R et al. The mechanism of action of aspirin. Thromb Res. 2003 Jun. 15; 110(5-6):255-8. doi: 10.1016/s0049-3848(03)00379-7). Similarly, proton pump inhibitors (PPIs) like omeprazole irreversibly block the gastric proton pump, resulting in acid suppression lasting more than 24 hours despite a plasma half-life of around one hour, an effect widely recognized in clinical practice (Sachs G., et al. Review article: the clinical pharmacology of proton pump inhibitors. Aliment Pharmacol Ther. 2006 June; 23 Suppl 2:2-8. doi: 10.1111/j.1365-2036.2006.02943.x.)

Another class where persistent pharmacodynamic effects are well-documented is corticosteroids. Acting through modulation of gene transcription, corticosteroids such as methylprednisolone induce or repress proteins that continue to affect biological processes long after plasma drug levels have declined. Animal studies demonstrate that gene expression changes can persist up to 72 hours following drug elimination, supporting the clinical observation of prolonged anti-inflammatory or immunosuppressive effects even after corticosteroid withdrawal (Barnes P J. How corticosteroids control inflammation: Quintiles Prize Lecture 2005. Br J Pharmacol. 2006 June; 148(3):245-54. doi: 10.1038/sj.bjp.0706736).

Antidepressants further illustrate the disconnect between plasma pharmacokinetics and clinical effects. These agents, including SSRIs and SNRIs, show a delayed onset of therapeutic action and can exert effects that persist after discontinuation. Neuroadaptive mechanisms, such as receptor regulation and epigenetic modifications affecting genes like BDNF, contribute to these lasting effects, reinforcing the notion that the therapeutic benefits of antidepressants are due to enduring changes in neuronal function rather than the immediate presence of the drug (Nestler E J. Epigenetic mechanisms of depression. JAMA Psychiatry. 2014 April; 71(4):454-6. doi: 10.1001/jamapsychiatry.2013.4291).

Finally, monoclonal antibodies and related immunotherapies present striking examples of prolonged biological effects that extend far beyond drug elimination. Rituximab, an anti-CD20 monoclonal antibody, depletes B cells for 6-12 months after administration, even though the antibody itself is cleared within weeks. Similarly, immune checkpoint inhibitors can trigger durable anti-tumour immune responses that persist after therapy ends, attributed to the establishment of immune memory (Sharma P., et al. The future of immune checkpoint therapy. Science. 2015 Apr. 3; 348(6230):56-61. doi: 10.1126/science.aaa8172).

In addition, also for conventional drugs, there are several examples of well-known persistent pharmacodynamic effects in conventional drugs which indicates that the conventional ADME provides only partial information on the drug's effects of persistence in the organism. These examples across diverse pharmacological classes highlight a critical point: the temporal relationship between plasma drug levels and biological activity is often complex and non-linear. Traditional ADME profiling, while extremely useful, captures only part of the reality of drug effects within living organisms. Understanding the persistence of pharmacodynamic actions is therefore crucial, especially when evaluating complex therapeutic approaches that rely on systemic reprogramming and physiological rebalancing rather than transient chemical presence.

All these examples highlight the “limitation” of rigidly correlating the duration of a substance's action with its plasma concentration or chemical half-life and illustrates the need for alternative analytical strategies capable of capturing the organism-level biological response to natural matrices-based products.

### SUMMARY OF THE INVENTION

The present invention provides an innovative analytical strategy specifically designed to overcome the inherent limitations of the traditional ADME (Absorption, Distribution, Metabolism, Excretion) approach in the pharmacokinetic and pharmacodynamic characterization of natural matrices-based therapeutic or beneficial product, such as a product comprising or consisting of multicomponent botanical extracts.

The invention provides a fundamentally different approach that is not based on the molecular profile (i.e., an approach independent of the standardisation of each and every “marker”) of the administered therapeutic or beneficial product. The approach proposed herein, captures the integrated, functional response of the organism to the administration of a therapeutic or beneficial product, i.e., relies on a methodology that characterizes the pharmacokinetic and pharmacodynamic behaviour of a therapeutic or beneficial product through functional biological response, independent of its chemical composition. This approach, also applicable to conventional drugs and beneficial products, is particularly suitable for multicomponent products, such as products comprising natural matrices, e.g., plant natural matrices, such as those derived from plant extracts, parts, fractions, or the like.

The functional evaluation of the organism's global biological response to the administration of the natural matrices-based therapeutic or beneficial product is achieved through a systematic, organ-specific, and time-resolved transcriptomic analysis, capable of capturing comprehensive gene expression profiles associated with both transient and persistent pharmacodynamic effects. This improvement on standard transcriptomic analysis provides a direct, quantitative, and integrative measure of the onset, duration, resolution of the biological activity (in terms of transcriptomic perturbations of genes expression) induced by the product under examination, regardless of the detectability of its original chemical constituents in plasma or other tissues. In addition, this improved method provides information on the trend in time of the magnitude of the transcriptomic perturbations underlying the biological activity.

Importantly, the method is applicable to any therapeutic or beneficial product, particularly to natural matrices-based products, i.e., products comprising or consisting of one or more natural matrices including but not limited to, plant extracts and/or parts, animal extracts and/or parts, natural sources of minerals such as eggshells, fossils, fungal extracts and/or parts and the like, where conventional ADME fails to capture the relevant information.

The analytical method of the present invention comprises:
1. Collection of biological samples from target organ samples (such as liver, kidney, brain) and blood at multiple predetermined time points post-administration obtained from healthy animal models as well as from sham control animals (i.e., animals undergoing the same experimental intervention of the treated group, such as same handling, same administration route and frequency, same sampling time points, etc., minus the active therapeutic component/s, e.g. an animal to which a placebo or vehicle is administered with the same administration route and timing of the product of interest) under standardized experimental conditions that underwent controlled administration of the natural matrices-based therapeutic or beneficial product or sham treatment;
2. Whole-transcriptome analysis of collected tissues to evaluate, for each tissue at each time point, gene expression changes over time, by means of array-based or sequencing-based platforms. This transcriptomic analysis thus detects changes in gene expression across key organs, yielding a “biological signature” of the product's effect;
3. Bioinformatic and statistical processing of the transcriptomic data to identify perturbation patterns, using tools such as:
   Differential Expression Analysis statistically significant changes in individual gene expression levels and, optionally,
   Molecular Degree of Perturbation (MDP) to quantify the global deviation of the transcriptome from a reference physiological state; and
4. Detecting and quantifying gene expression alterations associated with product exposure, by comparing transcriptomic profiles from product-treated animals with appropriate control groups, such as sham control animals and, optionally, untreated baseline animals;

Thereby identifying both transient effects (immediate response) and persistent effects (long-term biological imprinting) induced by the product under examination when compared to the controls in without need of tracking chemical markers.

### BRIEF DESCRIPTION OF THE DRAWINGS

FIG. 1—Molecular Degree of Perturbation in the Liver using all genes in the dataset: Molecular Degree of Perturbation (MDP) indicated as perturbation scores of Liver samples obtained from product treated and sham control treated mice, compared with samples from untreated control mice. The MDP is a single value per sample that reflects the overall transcriptional perturbation compared to an untreated control group. Analysis included all the available genes. Overall perturbation is represented by boxplots, with white boxes representing the untreated control group at timepoint 0, grey boxes representing the sham control group, and grid pattern boxes representing product-treated group. When comparing the product treated group with the sham control group there is a clear increase in the transcriptomic perturbation of product treated samples around 2 hours post-administration, which peaks at 6 hours and ends with basically no difference at 48 hours post-administration.

FIG. 2—Molecular Degree of Perturbation in the Liver using only ADME-related genes: Molecular Degree of Perturbation (MDP) indicated as perturbation scores of Liver samples from both EpigenAU/11 and sham control treated mice, compared with untreated control mice. The MDP is a single value per sample that reflects the overall transcriptional perturbation compared to an untreated control group. Analysis included only the ADME-related genes available in the state of the art (Dong Gui Hu et al. The Expression Profiles of ADME Genes in Human Cancers and Their Associations with Clinical Outcomes Cancers 2020, 12(11), 3369). Overall perturbation is represented by boxplots, with white boxes representing the untreated control group at timepoint 0, grey boxes representing the sham control group, and grid pattern boxes representing product-treated group. When comparing the product treated group with the sham control group there is an important increase in the transcriptomic perturbation of product treated samples around 6 hours post-administration, with an even better distinction of product treated and sham control treated mice. The perturbation gradually decreases and ends with basically no difference at 48 hours post-administration.

FIG. 3—Molecular Degree of Perturbation in the Kidney using all genes in the dataset: Molecular Degree of Perturbation (MDP) indicated as perturbation scores of kidney samples from both product treated and sham control treated mice, compared with untreated control mice. The MDP is a single value per sample that reflects the overall transcriptional perturbation compared to an untreated control group. Analysis included all the genes available in the Clariom™ S Pico Assay, Mouse (Applied Biosystems, ThermoFisher Scientific) indicated above. Overall perturbation is represented by boxplots, with white boxes representing the untreated control group at timepoint 0, grey boxes representing the sham control treatment group, and grid pattern boxes representing product-treated group. When comparing the product-treated group with the sham control group there is an important increase in the transcriptomic perturbation of product treated samples around 2 hours post-administration, which gradually decreases and ends with basically no difference at 48 hours post-administration.

FIG. 4—Molecular Degree of Perturbation in the Kidney using only ADME-related genes: Molecular Degree of Perturbation (MDP) of kidney samples from both product and sham control treated mice, compared with untreated control mice. The MDP is a single value per sample that reflects the overall transcriptional perturbation compared to an untreated control group. Analysis included only the ADME-related genes available in the state of art (Schlosser, P., Li, Y., Sekula, P. et al. Genetic studies of urinary metabolites illuminate mechanisms of detoxification and excretion in humans. Nat Genet 52, 167-176 (2020). Overall perturbation is represented by boxplots, with white boxes representing the untreated control group at timepoint 0, grey boxes representing the sham control treatment group, and grid pattern boxes representing product-treated group. When comparing the product-treated group with the sham control group there is an important increase in the transcriptomic perturbation of product treated samples around 6 hours post-administration, with an even better distinction of product treated and sham control treated mice. The perturbation gradually decreases and ends with basically no difference at 48 hours post-administration.

FIG. 5—Molecular Degree of Perturbation in the Blood using all genes in the dataset: Molecular Degree of Perturbation (MDP) of Blood samples from both product treated and sham control treated mice, compared with untreated control mice. The MDP is a single value per sample that reflects the overall transcriptional perturbation compared to an untreated control group. Analysis included all the genes available in the Clariom™ S Pico Assay, Mouse (Applied Biosystems, ThermoFisher Scientific) Overall perturbation is represented by boxplots, with white boxes representing the untreated control group at timepoint 0, grey boxes representing the sham control group, and grid pattern boxes representing product-treated group. There are no clear perturbation differences between product treated and sham control groups, with exception of the timepoint 18 hours in which samples from the sham control group presented a higher transcriptomic perturbation degree.

FIG. 6—Molecular Degree of Perturbation in the Brain using all genes in the dataset: Molecular Degree of Perturbation (MDP) of brain samples from both product and sham control treated mice, compared with untreated control mice. The MDP is a single value per sample that reflects the overall transcriptional perturbation compared to an untreated control group. Analysis included all the genes available in in the Clariom™ S Pico Assay, Mouse (Applied Biosystems, ThermoFisher Scientific) as defined above. Overall perturbation is represented by boxplots, with white boxes representing the untreated control group at timepoint 0, grey boxes representing the sham control group, and grid pattern boxes representing product-treated group. When comparing the product-treated group with the sham control group there is an increase in the perturbation degree at 2 hours post-administration, which is also observed in samples from other organs like Liver and Kidney. At 24 hours it is also possible to see differences between these groups, which seem to decrease at 48 hours,

FIG. 7—Line plot with Number of Differentially Expressed Genes in the Liver: Number of genes in which the expression in the product treated group is statistically different (adjusted p-value<0.05) from the expression in the sham-control group. The continuous line in grey represents the total number of up-regulated genes (treated group expression is higher compared to sham control group) in each time point, and in black represents total number of down-regulated genes. Dashed lines, on the other hand, represent the number of differentially expressed genes that are ADME-related, following the same colour code (grey up and black down regulated). There is indeed a peak of DEGs at 6 hours post-administration, with very few DEGs at 48 hours (with no evidence of significant perturbation of ADME-related genes).

FIG. 8—Line plot with Number of Differentially Expressed Genes in the Kidney: Number of genes in which the expression in the product-treated group is statistically different (adjusted p-value<0.05) from the expression in the sham-control group. The continuous line in grey represents the total number of up-regulated genes (treated group expression is higher compared to sham control group) in each time point, and in black represents total number of down-regulated genes. Dashed lines, on the other hand, represent the number of differentially expressed genes that are ADME-related, following the same colour code (grey up and black down regulated). There is a peak of DEGs at 2 hours post-administration, with very few DEGs at 48 hours (with no evidence of significant perturbation of ADME-related genes).

FIG. 9—Line plot with Number of Differentially Expressed Genes in the Blood: Number of genes in which the expression in the product-treated group is statistically different (adjusted p-value<0.05) from the expression in the sham-control group. The grey line represents the total number of up-regulated genes (treated group expression is higher compared to sham control group) in each time point, and in black represents total number of down-regulated genes. There is a peak of DEGs at 0 hours post-administration, which might reflect an instant effect on the blood transcriptome. A second peak in the number of DEGs occurs at 18 hours post-administration, also influenced by the higher perturbation in the sham control samples observed in the Molecular Degree of Perturbation analysis.

FIG. 10—Line plot with Number of Differentially Expressed Genes in the Brain: Number of genes in which the expression in the product-treated group is statistically different (adjusted p-value<0.05) from the expression in the sham-control group. The grey line represents the total number of up-regulated genes (treated group expression is higher compared to sham control group) in each time point, and in black represents the total number of down-regulated genes. There is a peak of DEGs at 2 hours post-administration, which follows the same pattern observed in other organs such as Kidney and Liver. The perturbation abruptly decreases with few DEGs at 6 hours post-administration, and no significant perturbations at 48 hours.

FIG. 11—Line plots with Number of Differentially Expressed Genes from Physiological Solution treated samples against untreated samples at timepoint 0 h: Number of genes in which the expression in the sham-control group is statistically different (adjusted p-value<0.05) from the expression in untreated control group. The grey line represents the total number of up-regulated genes (sham-control group expression is higher compared to untreated control group) in each time point, and in black represents the total number of down-regulated genes. Blood shows similar peaks of perturbation at 0 and 18 hours. Brain presented a few down-regulated genes at 18 hours. Kidney had some DEGs perturbed at 6 hours, with a peak at 12 hours followed by a decrease in the perturbation. In the liver, some genes are statistically perturbed from 6 to 18 hours after treatment. The figure shows that the sham treatment per se induces perturbation, therefore the correct transcriptomic comparison is between product treated and sham control groups, and that also circadian cycle influences transcriptomic perturbation therefore the same time points between product and sham treated groups must be compared.

FIG. 12 Area Under the Effect Curve (AUEC). The figure shows the sum of all different biological samples tested (i.e., liver, kidney, brain, blood) see FIGS. 1-10 and examples) transcriptomic perturbations induced by the product under examination (Epigen11/AU) over time (hours). Dots represent the measurements sum at each timepoint. x-axis represents the time (hours) and y axis the overall perturbation (total number of DEGs). The grey shadow represents the AUEC.

### GLOSSARY

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular.

In any point of the present specification or of the claims, the expression “comprising” or “comprise(s)” can be replaced by “consisting of” or “consist(s) of”.

A “natural matrix” in the present application refers to a material consisting of a network represented by a broad number of components/constituents obtained (e.g. extracted) directly from a member of the natural kingdom or a naturally occurring portion thereof (i.e., from a natural raw source), without significant processing or synthetic alteration, wherein “without significant processing or synthetic alteration” is intended that no denaturing processes are used for obtaining the matrix from the raw source. In other words, the natural raw source is processed only by manual, mechanical or gravitational means e.g. by dissolution in water or other naturally occurring solvents, such as water, water-alcohol solutions etc.; by flotation; by extraction with water or other naturally occurring solvents; by steam distillation or by heating solely to remove water or any other naturally occurring solvent; or extracted from air by any means and with the provision that “natural matrix” excludes said member of the natural kingdom “as such” i.e. non-processed. According to the invention, a natural matrix is a 100% natural and biodegradable material, consisting of natural components that have not been denatured by the process to produce of the matrix from the starting raw materials without intentional addition of synthetic products along the whole process. In the present description, 100% biodegradable is considered as “readily biodegradable” according to an OECD biodegradability test. These features guarantee the maintenance of the matrix effect which is conferred to the matrix by the presence of structural interactions by its components (material interactions) and functional interactions that become evident upon exposure of a biological system to the natural matrix (immaterial interactions). In other words, a natural matrix, or a mixture of natural matrices, is a material obtained from entities that are self-assembled in nature and processed so to preserve their native bio-physical characteristics which determine their physiological interaction with other living organisms, such as the human organism. Their emerging properties can be expressed by contributing to the rebalancing of metabolic processes or states of the receiving organism and/or of some organs or tissues alongside the physiological actions that will be activated in each specific context. According to the present invention the natural matrix can be from a material obtained from any source in the life kingdoms i.e., Monera, Protista, Fungi, Plantae and Animalia. The term hence encompasses a plant natural matrix, an animal natural matrix, a fungi natural matrix, a Protista (archaea or bacteria) natural matrix, a Monera natural matrix. A natural matrix may also comprise natural inorganic materials such as minerals obtained from natural raw materials. A synonym of natural matrix or one or more natural matrices in the present description is “complex natural system” or “natural material” as defined below.

An example of naturally occurring portion of an organism may be represented by e.g., roots, leaves, bark, fruit, flower, of a plant or sections thereof, organs, tissues.

In any part of the description the general term natural matrix can be substituted with:
a plant natural matrix or a natural matrix obtained from a plant,
an animal natural matrix or a natural matrix obtained from an animal or from an animal product such as eggs or milk,
a fungi natural matrix or a natural matrix obtained from a fungus,
a Protista natural matrix or a natural matrix obtained from a Protista,
a Monera natural matrix or a natural matrix obtained from a Monera,
or with a plant material and/or extract, an extract from an animal tissue or organ, fungi and/or a fungi extract, or a mixture thereof wherein the extraction process does not encompass denaturing steps (e.g., temperature or the use of denaturing solvents).

Plant is synonymous with herb.

The term “natural” matrix emphasizes the retaining the integrity and complexity of networks of constituents/components as in the original natural source due to the absence of denaturing treatments for the obtainment thereof. A natural matrix hence does not encompass compositions of natural origin that are enriched in specific molecules of artificial synthesis or isolated from a natural raw material. In addition, a natural matrix is obtainable only with processes that do not act through extensive processing or chemical modification, isolation, purification, or molecular extraction.

Due to the supramolecular self-assembly of the constituents/components of a natural matrix and the presence of functional interactions among them, the whole matrix behaves as a complex network that does not interact with a single target molecule but that interacts with a network of recipients (also organised as a network) in the receiving organism. Therefore, the interaction between the natural matrix and the receiving organism is not, as for common pharmaceutical APIs the result of a point-to-point interaction, but the result of an “interactor” networks (i.e., the matrix)-“receiver” network (i.e., the organism to whom the matrix is administered) interaction.

The term natural matrix can be also substituted in any part of the description and of the claims with the term is “complex natural system” or “natural material”.

“Emerging properties” according to the present description and to the art, the term defines the properties of a natural matrix or of a natural material according to the present specification, i.e., properties that are not represented by the mere sum of properties of each singled out constituent/component of said matrix/material but by both functional and structural interactions among all constituents/components of the matrix/material that are also the result of the supramolecular self-assembly of said components/constituents within the matrix/material itself.

“Emerging properties” hence refer to technical effects, such as therapeutic or homeostasis-adjuvating properties (i.e., beneficial effect), that the interactions and relationships among the constituents/components of a natural matrix exert on a receiving living system. Emergent properties are properties that are not immediately evident or even predictable based solely on the individual characteristics of each constituent/component of the matrix. Instead, they “emerge” when all the constituents/components of the matrix networks interact with one another and with the living system receiving network in a dynamic and complex way. Emerging properties have been broadly discussed in the art in various scientific and systems-oriented fields, including physics, chemistry, biology, and complex systems theory. Emerging properties are hence properties that cannot be predicted a priori by the quali-quantitative knowledge of each component of a given composition or matrix and that, consequently, cannot be ascribed to one or more specific API. Hence, although multidrug compositions can show unpredicted synergic effects, the properties of said compositions are still ascribed to the specific APIs and quantities thereof contained therein.

In the case of emerging properties, characteristic of natural matrices, the observed emerging properties cannot be attributed to specific APIs and are maintained in different batches of a given matrix or a given mixture of matrices notwithstanding the different quali-quantitative composition of said batches (functional resilience see below).

“Synthetic” according to the present description has the meaning conventionally accepted in chemistry. Conventionally, in chemistry, the term “synthetic” refers to the origin or source of a material or substance. Synthetic substances or materials are produced by man through artificial synthesis i.e., through laboratory chemical reactions usually by reacting simpler chemicals to create more complex ones through processes that often use different pathways, temperature conditions, pressure conditions, energy sources and/or catalysers from those used by living organisms. Examples: Synthetic substances or materials include plastics, pharmaceutical drugs, and many industrial chemicals. For example, nylon is a synthetic polymer made through chemical synthesis, and aspirin is a synthetic drug produced through specific chemical reactions.

“Functional resilience” (also indicated as “redundancy”) according to the present description is intended as a therapeutic or beneficial (homeostasis adjuvant) resilience of a therapeutic or beneficial product comprising or consisting of one or more natural matrices; the term describes the maintenance of the therapeutic or beneficial properties of different batches of a given product comprising (or consisting of one or more natural matrices) notwithstanding the different batch to batch qualitative and quantitative composition, which is necessarily present (inherent) in products comprising or consisting of one or more natural matrices. As known by the skilled person, each time a different batch of starting raw material is used, the resulting natural matrix has a unique quali-quantitative composition at the molecular level which is typical of the individual diversity between living organisms also of the same species.

A “healthy physiological state” refers to the condition of an organism's body, organ, apparatus, system or body region, and its internal processes when they are functioning optimally and within normal parameters for that individual, i.e., the state to which homeostasis tends. A healthy physiological state, in the context of one or more biological activities known to contribute to hallmarks of a given disease or pathological condition or of an altered physiological state, refers to the state in which said one or more biological activities are operating optimally and within normal (healthy) parameters. This state is characterized by the absence of significant aberrant cellular or molecular processes associated with the specific disease under consideration. When the modification trend of one or more biological activities which concurs to a pathological, pre-pathological condition is known, the healthy physiological state can be considered represented by the opposite modification trend for each of said activities. The term considers the hallmarks of a particular disease, which are distinctive features or characteristics that are typically observed in individuals affected by that disease. These hallmarks can include specific cellular behaviours, molecular pathways, canonical pathways, or physiological responses that play a key role in the development or progression of the disease.

In summary, a healthy physiological state in the context of a specific disease or pathological/altered condition is a state in which the one or more biological activities related to the known hallmarks of that disease or pathological condition are modulated in a direction that is consistent with a non-pathological/non-altered state, in other words, opposed to the pathological/altered state.

A healthy physiological state according to the invention, therefore, also indicates the direction of the modulation of one or more biological activities that are known hallmarks of a pathological condition in homeostasis, i.e., before the onset of a pathological condition, in other words the homeostatic direction of the modulation of one or more biological activities ascribed to a specific system, district, apparatus or organ of a healthy subject.

“Altered physiological states” and “altered homeostasis” are closely related concepts that describe deviations from the normal functioning and balance of the body's internal environment. While they overlap, there are some distinctions between the two terms:
Altered Physiological States: This term encompasses a broad range of changes in the body's normal functioning, including disruptions in organ systems, biochemical processes, and cellular functions. Altered physiological states can result from various factors such as disease, injury, medication, environmental factors, and psychological stress. Examples include fever, inflammation, hormonal imbalances, and impaired organ function.
Homeostasis (altered): Homeostasis refers to the body's ability to maintain a stable internal environment despite external or pre-pathological changes. This stability is achieved through regulatory mechanisms that control variables such as body temperature, blood pressure, pH balance, and blood glucose levels within narrow ranges. Altered homeostasis occurs when these regulatory mechanisms fail to maintain balance, leading to deviations from the body's normal set points. These deviations can be temporary or chronic and may involve compensatory mechanisms to restore balance.

In summary, altered physiological states describe the observable changes in the body's normal functioning, while altered homeostasis refers to the underlying disruption of the body's regulatory mechanisms that maintain internal stability.

An altered homeostasis underlies altered physiological states, as disruptions in homeostatic mechanisms that can lead to physiological imbalances and manifestations of illness or dysfunction. A product adjuvating homeostasis is a product that adjuvates the body to restore the stability of its internal environment when altered.

“Hallmark of a disease” or of a pathological or medical condition according to the present description has the meaning conventionally used in the art. Hallmarks of a disease are known to be indicators that can mark the progression or control of a given disease or pathological or pre-pathological condition and taken together are usually representative of the general pathological state associated to a given pathology. These hallmarks (also called ‘key indicators’) are typically a set of features or patterns that a physician would monitor, over time, to track the onset, the progression or regression of a particular illness. In summary, a hallmark of a disease is a defining feature or characteristic whose modification is indicative of a given pre-medical or medical condition, aiding in its identification, diagnosis, monitoring and understanding. By way of example, for neurodegenerative diseases (NDDs) at least the following eight hallmarks of NDD are known in the art: (pathological protein) aggregation, synaptic and neuronal network (dysfunction), (aberrant) proteostasis, cytoskeleton (abnormalities), (altered) energy homeostasis, DNA and RNA (defects), inflammation (increase), and neuronal cell death (increase). By way of example, in cancer research, the hallmarks of cancer are a set of distinctive characteristics that are commonly found in cancer cells. These hallmarks include (sustained) proliferative signalling, (evasion of) growth suppressors, (resistance to) cell death, (enabling) replicative immortality, (inducing) angiogenesis, and (activating) invasion and metastasis. Hallmarks of a disease, parameters related to said hallmarks (e.g., biomarkers), one or more biological activities associated to said hallmarks etc. are a framework to study a disease or a pathological or medical condition using an integrated/holistic approach.

The hallmarks of an altered physiological state typically include observable changes in various aspects of the body's functioning, which may manifest through symptoms, signs, or laboratory findings.

Altered physiological states typically reflect disruptions in the body's homeostatic mechanisms, leading to deviations from normal physiological parameters. These imbalances may involve alterations in temperature regulation, fluid and electrolyte balance, acid-base balance, glucose metabolism, or other regulatory processes.

Overall, the hallmarks of an altered physiological state provide valuable clues for healthcare providers to identify the underlying cause, assess severity, and guide appropriate interventions to restore normal functioning and promote recovery.

Native natural intelligence, represents the intrinsic ability of natural matrices to conserve and transmit biological and physical-chemical information necessary for interacting and integrating with other living networks, using logics inherent to the living organism that receives them, as they are already known to it, and therefore endogenous in relation to it. This intelligence is an expression of natural autopoiesis, that is, the ability to self-organize and adapt to environmental stimuli without artificial intervention, which would transmit a message according to point-like logics and through mediators unknown to the living organism receiving them, and therefore exogenous in relation to it.

The expression Physiological Interconnection, defined as “endogenous” physiological interconnection, describes the ability of a natural matrix to interact in a harmonious and functional way with the biological systems of the recipient based on the fact that they both belong to the domain of what is living (endogenous), stimulating internal responses to restore balanced physiological states. This interaction is based on natural dynamics, without artificial interventions, and represents a reciprocal dialogue between the matrix and the organism, promoting self-regulation and physiological recovery.

Self-assembled entities in nature defines complex systems made up of multiple components that spontaneously organize into functional structures through chemical-physical interactions that occur in natural environments and conditions. These systems, found in living organisms or natural matrices, exhibit emergent properties that arise from their dynamic interactions and cannot be replicated artificially.

When referring to a subject in need of a beneficial or therapeutic treatment, the description relates to a human being, either affected by a pathological condition or at risk of developing a pathological condition.

ADME is used herein in the meaning commonly used in the art, is an acronym that stands for Absorption, Distribution, Metabolism, and Excretion, and describes the fundamental pharmacokinetic processes that determine the fate of a drug or other substance within a living organism following its administration. It encompasses the entry of the substance into the bloodstream (absorption), its dissemination throughout body fluids and tissues (distribution), its chemical transformation by enzymatic systems primarily in the liver (metabolism), and its elimination from the body, typically through renal or biliary pathways (excretion). ADME studies are critical in pharmaceutical development for understanding a substance's bioavailability, pharmacokinetic c profile, efficacy, toxicity, and appropriate dosing strategies.

Product A or EpigenAU/11 according to the present description is as natural matrices based therapeutic product as defined in the examples section.

### Definition of Conventional ADME Pharmacokinetic Parameters:

In the context of **ADME** (Absorption, Distribution, Metabolism, and Excretion), the following pharmacokinetic parameters are commonly used to describe how a drug behaves in the body:
Cmax (Maximum Concentration) definition: The highest concentration of a drug in the bloodstream after administration. Cmax Indicates the peak exposure of the drug and is often related to the intensity of its pharmacological effect or potential toxicity.
Tmax (Time to Maximum Concentration) definition: The time it takes to reach Cmax after drug administration. Tmax Reflects the rate of absorption; a shorter Tmax means the drug is absorbed more quickly.
AUC (Area Under the Curve) definition: The area under the plasma concentration-time curve, representing the total drug exposure over time, used to assess the extent of absorption and*bioavailability. The larger the AUC, the more drug has entered systemic circulation.
MRT (Mean Residence Time): which reflects the average duration in which a molecule remains in the system

When preceded by the letter f, the acronyms above refer to the equivalent information in terms of functionality, i.e., transcriptomic perturbation induced by the product tested.

Clearance (CL) definition: The volume of plasma from which the drug is completely removed per unit time (e.g., mL/min or L/h), reflecting the efficiency of drug elimination through metabolism and excretion. It is crucial for calculating maintenance dose.

According to the invention, the respective ADME-equivalent parameters Vmax/transcriptomic Peak, functional Tmax, AUEC (Area Under the Effect Curve), Functional half-life (or Mean Residence Time) and resolution time, provide similar information that can be used for the same purposes. In the present description and claims, a sham control animal follows the official definition in scientific literature, i.e., it is an animal that undergoes a control intervention that mimics all aspects of the experimental procedure, such as anaesthesia, surgical exposure, handling etc., except for the critical therapeutic or experimental product/component being tested. The sham group serves as a control to isolate the specific effects of the experimental treatment from general procedural effects. In summary, a sham procedure in animal trials is a control method designed to replicate all aspects of an experimental intervention, minus the active component/product, to accurately assess the intervention's specific effects.

This glossary is intended as basis for claims or description rewording or clarification.

### DETAILED DESCRIPTION OF THE INVENTION

As described above, the present invention relates to an improved transcriptomic-based method for functionally characterising the onset, duration, and resolution of the biological effects of a medicinal product following its administration to a subject. The method characterizes therapeutic or beneficial products and is particularly suitable for natural matrices-based products, i.e., compositions of matter consisting solely of natural matter, exerting a therapeutic or beneficial effect, and having a physiological mode of action.

The conventional ADME (Absorption, Distribution, Metabolism, Excretion) framework, based on a reductionist and molecule-centred strategy, assumes that the biological effect of a pharmacological be predicted, monitored, and understood by isolating one or more chemical constituents, quantifying them in biological matrices (plasma, tissues), and correlating their concentration-time profile with pharmacodynamic effects. Techniques such as HPLC, GC-MS, and LC-MS/MS are typically used to define standard pharmacokinetic parameters, including half-life, Cmax, Tmax, AUC, bioavailability, clearance, and volume of distribution. As stated above, while highly effective for chemically homogeneous drugs containing a single active principle, this approach becomes fundamentally inadequate, if not misleading, when applied to natural matrices-based products such as multicomponent botanical extracts or products comprising the same.

Natural matrices, such as, e.g., plant extracts, are inherently variable and complex mixtures of structurally diverse secondary metabolites-polyphenols, flavonoids, terpenoids, alkaloids, glycosides, and others-whose physicochemical properties vary widely. Consequently, as well-known in the art, no single chemical “fingerprint” can adequately represent the entire extract or its biological effect; Many minor or unstable constituents may lack validated analytical standards, making their precise quantification impossible and, the presence of matrix effects (interferences from other components of the extract) frequently complicates or prevents accurate measurement, even when advanced analytical platforms are employed.

Furthermore, conventional analytical techniques typically focus on a pre-selected subset of “marker” compounds, chosen based on chemical abundance or ease of detection, rather than on biological relevance. This introduces an intrinsic bias and may lead to an incomplete or distorted assessment of the extract's true pharmacokinetic and pharmacodynamic profile.

A fundamental limitation of the molecular approach (e.g., ADME) is its inability to capture the functional and emergent properties of natural matrices-based products. As known, natural matrices, such as, e.g., plant extracts, do not act as simple sums of their individual constituents; rather, they exert biological effects through multi-layered interactions such as synergistic interactions (where multiple molecules enhance each other's bioavailability, stability, or pharmacodynamic impact); antagonistic interactions, (where certain components modulate or limit the activity of others, contributing to a balanced pharmacological profile) and, sequential and complementary mechanisms, (where different compounds act on distinct but interconnected molecular targets or pathways).

These multi-layered interactions normally lead to pharmacodynamic outcomes that are not predictable based on the concentration of any single component or detectable metabolite. Moreover, some bioactive effects may be mediated by secondary metabolites generated in vivo through metabolic transformation, microbiota activity, or conjugation processes, further complicating any attempt at direct analytical correlation. A further critical limitation of the traditional approach is the temporal dissociation between the pharmacokinetics of detectable constituents and the duration of biological effects. In many cases, the therapeutic or physiological impact of a natural matrices-based product persists well beyond the time window in which the original compounds, or their known metabolites, are detectable in plasma or tissues. This may result from tissue accumulation of certain compounds or their derivatives, with delayed release or activity; epigenetic modifications, transcriptional reprogramming, or other long-lasting biological adaptations triggered by short-term exposure and modulation of endogenous regulatory systems (e.g., immune, endocrine, or metabolic pathways) that remain altered after the compound has been metabolized and cleared.

Under these circumstances, conventional ADME metrics fail to provide a reliable estimate of the extract's biological persistence, therapeutic efficacy, or safety profile. The analytical and conceptual challenges described above are not hypothetical; they are routinely encountered in the pharmacokinetic assessment of numerous natural matrices. Typical examples include botanical natural matrices containing both rapidly cleared and long-lasting constituents such as, e.g., flavonoids and terpenes that often exhibit markedly different half-lives, leading to complex, asynchronous concentration-time profiles. By way of example, several studies have shown that closely related natural compounds within the same plant matrix may exhibit significantly different pharmacokinetic properties. especially elimination half-lives, despite structural similarity. Furthermore, naturally occurring metabolites can frequently be metabolized to endogenous metabolites. This reinforces the idea that tracking a single molecule does not adequately represent the pharmacokinetic behaviour of an entire plant matrix. Notable examples include flavonoids (e.g., anthocyanins vs. flavonols) wherein polyphenol-rich extracts, anthocyanins and catechins (e.g., in berries or green tea) typically exhibit t½ of 2-3 Hours, while structurally similar flavonols like quercetin or myricetin may persist for 10-20 hours (Hollman, P. C. H. (2004). Absorption, Bioavailability, and Metabolism of Flavonoids. Pharmaceutical Biology, 42(sup1), 74-83. https://doi.org/10.3109/13880200490893492); triterpenoid saponins (Saikosaponin A vs. D) wherein Saikosaponin A and D differ only slightly in glycoside position, yet comparative pharmacokinetic studies show elimination half-lives of ~4.5 hours for Saikosaponin A and ~8.2 hours for Saikosaponin D in rats, with the D form consistently showing longer persistence (Xu L., et al. Analysis of saikosaponins in rat plasma by anionic adducts-based liquid chromatography tandem mass spectrometry method. Biomed Chromatogr. 2012 July; 26(7):808-15. doi: 10.1002/bmc.1734); and phthalides (Z-ligustilide vs. Levistilide A) wherein the half-lives of Z-ligustilide and its close analogue Levistilide A differ significantly, i.e., ~14 hours for Z-ligustilide and ~24 hours for Levistilide A (Liu, X et al. Comparative Pharmacokinetics Research of 13 Bioactive Components of Jieyu Pills in Control and Attention Deficit Hyperactivity Disorder Model Rats Based on UPLC-Orbitrap Fusion M S. Molecules. 2024 Mar. 10; 29(6):1230. doi: 10.3390/molecules29061230).

In addition, several botanical natural matrices generate bioactive metabolites in vivo undergoing metabolic transformation by liver enzymes or intestinal microbiota, producing compounds with greater stability, higher bioactivity, or distinct pharmacological properties compared to the original constituents, leading to biological effects that persist long after measurable compounds have disappeared from plasma or tissues. Furthermore, there is the objective lack of purified reference standards, in fact, several minor components of natural matrices with potential biological relevance are commercially unavailable or analytically intractable, precluding their quantification and pharmacokinetic profiling.

In such contexts, the absence of measurable plasma levels of one or more “marker” compound does not necessarily indicate the absence of pharmacodynamic effects. Under such conditions, conventional ADME parameters, when applied to natural matrices, would provide a misleading and certainly incomplete picture of pharmacodynamic reality. Real-world examples confirm these challenges. Botanical extracts often contain compounds with radically different half-lives, generating asynchronous concentration profiles.

Finally, variability in botanical source, environmental factors, harvesting methods, and processing conditions lead to substantial batch-to-batch differences in chemical composition thus rendering ADME analysis inappropriate ab initio for natural matrices based therapeutic or beneficial products. The idea of fixing a molecular fingerprint to define pharmacokinetics or predict clinical outcomes across batches of said kind of products is thus fundamentally flawed.

Surprisingly, as directly evident from FIG. 11, the inventors have found that in the experimental design of the method, samples from untreated animals are not suitable as controls for analysing transcriptomic perturbations as sham treatment is sufficient to alter transcriptomics, therefore, the transcriptomic data of samples from treated animals is, and must be, compared with the transcriptomic data of the corresponding sham control animals. Indeed, the figure shows that, compared to untreated animals, sham control animals show transcriptomic alterations following the sham administration that are not present in the untreated control, and that, with respect to the untreated control at to, transcriptomic alterations that are also influenced by the animals life circadian cycle are also observed. It follows that, for reliable and informative Differential Expression analysis, product treated vs untreated does not allow exclusion of Differentially Expressed Genes (DEGs) induced by the experimental design itself, and, in addition, that the comparison between product treated and sham treated group has to be performed at more than 1 time point in order to define the onset and the resolution of said perturbation. Multiple time points allow to provide further relevant information such as the exact resolution time, the duration of the perturbation and the ADME-equivalent parameters depicted in the table below.

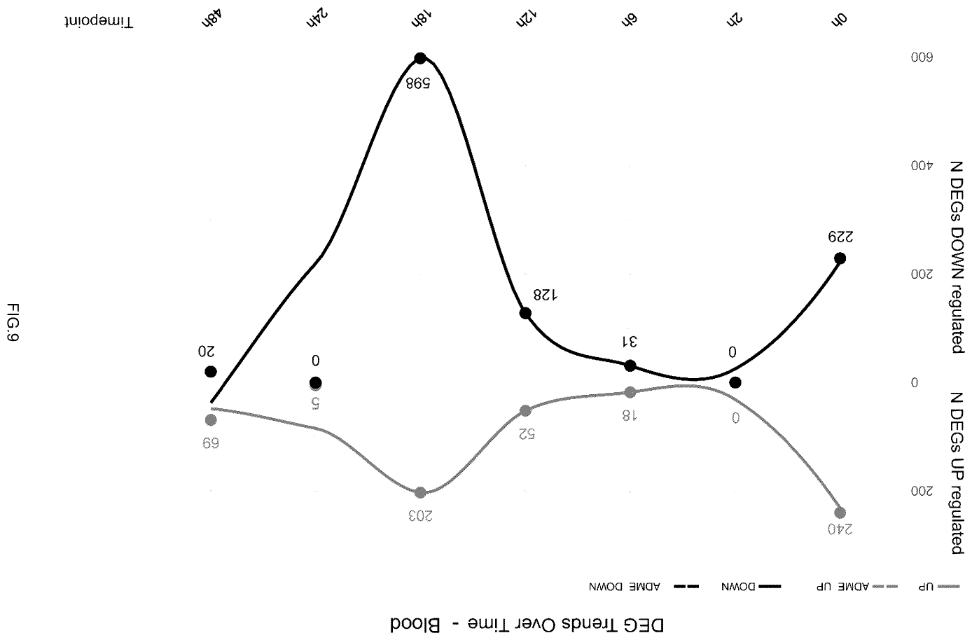

In addition, the inventors also found that, for these organs for which ADME related genes are disclosed in the art (e.g., liver and kidneys), the transcriptomic analysis of DEG ADME related genes, is not sufficient to provide the desired functional characterisation of the onset, duration and resolution of the biological effects of the analysed therapeutic or beneficial product (preferably natural matrices-based product) provided by the method of the invention. Indeed, as clear from FIGS. 7 and 8 the sole analysis of ADME related genes, while providing interesting and useful information such as, e.g., information on secretion time when kidneys ADME related genes are analysed fail to provide a significant line plot of relevant up and down regulation of the genes induced by the administration of the therapeutic or beneficial product under examination.

However, the detailed description and example will show that ADME related genes (when available in the art) expression analysis can provide additional relevant information.

Object of the present invention is a
method for functionally characterising the onset, duration and resolution of the biological effects of a therapeutic or beneficial product, said product preferably comprising one or more natural matrix, following its administration to a subject, the method comprising:
(a) extracting RNA from biological samples of different tissues obtained from living organisms at multiple time points following administration of said natural matrices-based product, hereinafter treated sample(s), and from corresponding biological samples obtained at the same time points from a sham control living organism (e.g., same administration and handling protocol with vehicle/placebo), hereinafter sham control sample(s);
(b) performing a transcriptomic analysis comprising gene expression profiling on the extracted RNA from each of said treated and sham control samples; and identifying, for each treated sample at each time point, transcriptomic perturbations in terms of significantly Differentially Expressed Genes (DEGs) relative to the corresponding sham control sample;
(c) defining the onset, duration, and resolution profile of the biological activity induced by the administration of said product
wherein
the onset time point corresponds to the first time point at which a significant transcriptomic perturbation is observed in at least one of said treated samples with respect to the corresponding sham sample;
the resolution time point corresponds to the last time point at which a significant transcriptomic perturbation is observed in said treated samples with respect to the corresponding sham samples;
the duration corresponds to the time span between said onset and resolution time point;

Additionally, the method of the invention allows to provide ADME-equivalent functional parameters as indicated in the table below:

| ADME | | | |
|---|---|---|---|
| CLASSIC | FUNCTIONAL | MEASURE | |
| PARAMETER | EQUIVALENT | PROVIDED | APPROACH |
| Cmax | Transcriptomic | Maximum intensity | All-Organs or |
| | peak, Pmax | of the therapeutic | organ specific |
| | | or beneficial | measurement |
| | | product effect | |
| Tmax | Functional Tmax | Promptness of the | All-organs or |
| | (fTmax) | response to the | organ specific |
| | | administration of | measurement |
| | | the therapeutic or | |
| | | beneficial product | |
| AUC | AUEC (area | Overall volume of | All-organs or |
| | under the | the effect | organ specific |
| | effect curve) | (intensity × | measurement |
| | | duration) induced | |
| | | by the | |
| | | administration of | |
| | | the therapeutic or | |
| | | beneficial product | |
| MRT (Mean | Functional Mean | Mean time of the | All-organs or |
| Residence | Residence Time | system (i.e., | organ specific |
| time) | (fMRT) | organism) | measurement |
| | | perturbation | |
| | | following | |
| | | administration of | |
| | | the therapeutic or | |
| | | beneficial product | |
| Clearance | Resolution time | Reinstatement of | All-organs or |
| | fTres | the basal | organ specific |
| | | transcriptome (end | measurement |
| | | of transcriptional | |
| | | perturbation) | |
| | | induced by the | |
| | | administration of | |
| | | the therapeutic or | |
| | | beneficial product | |

The product's specific Pmax for a given organ defines the maximum number of DEGs observed in the treated samples obtained from a given organ and Pmax calculation can be extended at the systemic level.

The product's fTmax for a given organ is the time point at which the highest significant transcriptomic perturbation peak observed in the treated samples obtained from a given organ whereas the overall product's fTmax corresponds to the time point at which the maximum overall number of significant transcriptomic perturbations is observed among all treated samples from all organs;

The product's AUEC is the numeric integration of the DEGs or MDPs area vs. time.

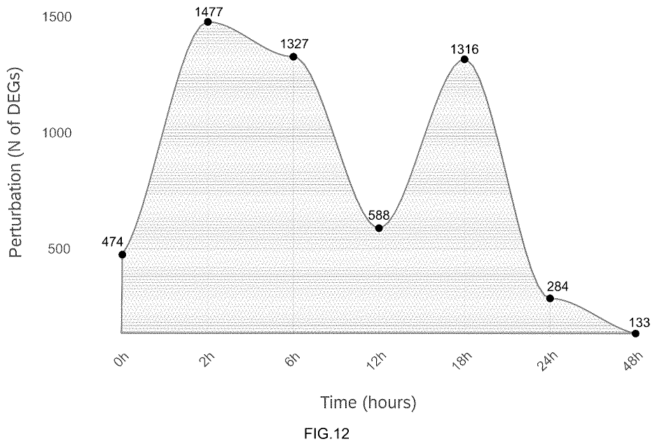

The pharmacokinetic concept of Half-life indicates the time required for the concentration to decrease by half, and has no direct equivalent in transcriptomic responses, as these do not typically decay in a linear manner (It is possible to see in FIG. 12 that the initial peak is followed by a second, less intense, peak). Therefore, a more suitable analogue is the “Functional Mean Residence Time (fMRT)”, which reflects the average duration in which a transcriptomic perturbation remains in the system. In the art, MRT is calculated by the AUMC/AUC ratio, according to the formula provided in the detailed description, resolution time is the first time point in which there is no more significant transcriptomic perturbation (end of perturbation), according to the invention, fMRT is calculated as AUMC/AUEC ratio.

Organ specific resolution time fTres corresponds to the time point at which reinstatement of the basal transcriptome (end of transcriptional perturbation, defined as a transcriptomic perturbation≤5% of the Pmax for all tissue samples excluded blood wherein the end of transcriptomic perturbation is defined as ≤12% Pmax, preferably ≤5% Pmax) induced by the administration of the therapeutic or beneficial product. As can be expected, transcriptomic perturbation in blood samples is more unstable as blood stream is more susceptible to alterations also due to variations such as glycaemic peaks immediately after feeding, day-night alternations and the like. When considered under a systemic point of view, fTres is the first time point at which all organs tested reach organ specific a transcriptomic perturbation≤5% of the Pmax for excluded blood wherein the end of transcriptomic perturbation is defined as ≤12% Pmax, preferably ≤5% Pmax.

Reference to Organ-specific or All-organs in the table above, means that, depending on the desired information, the ADME-like functional parameters of the invention can be calculated on samples obtained from a specific treated organ of interest or from a systemic point of view (“all-organs”), using the overall DEGs information obtained from all the samples tested, i.e., from the overall data on the samples of each organ. Details on the calculation of each of the parameters above are provided in the detailed description and in the examples.

As clear from the figures, the method of the invention provides organ specific line plots depicting the onset, duration, and resolution time frame of the biological effects (represented by measurable transcriptomics perturbations) induced by the administration of the tested product. The organ specific data can then be compared and elaborated so to provide information at the organism level. In other words, the method of the invention provides a biological persistence and clearance profile of said product based on organism-level functional transcriptomic responses, independent of direct quantification of individual chemical constituents thereof. The organism-level profile provided by the method of the invention is given by the concomitant analysis of the transcriptomic perturbation in an organism-representative selection of tissues. In addition, the analysis of the transcriptomic perturbation data obtained from either the organism-representative samples or from selected organ tissues samples, according to the invention, can provide ADME-equivalent functional parameters summarised in the table above.

The method of the invention advantageously enables developers and users to understand the actual duration of the activity and presence of therapeutic or beneficial product from the moment of its administration to a subject (even when the original compounds are in principle no longer measurable in plasma which are data not obtainable with a classic ADME evaluations) and to obtain reliable indications, about any persistent or delayed potentially toxic effects. In other words, the method of the invention allows to provide a reliable measurement of the effect of a therapeutic or beneficial product, when administered to a subject, even when classical ADME evaluation is not technically and economically possible.

Indeed, it is to note that, and ADME evaluation of a product whose active compounds cannot be determined, such as a product based on natural matrices, would require an exact determination of each and every compound within the product, and, due to the fact that “active compounds” cannot be determined, the ADME evaluation for each and every of said compounds. As in natural matrices such as plant extracts and the like, the compounds within the matrix are averagely hundreds or thousands, and the cost for a complete ADME would be unreasonable. Indeed, a market analysis has estimated that preclinical pharmacology studies, including ADME/PK profiling, account for approximately 10% of the total preclinical development budget for a new drug. Given that the average total preclinical investment is around $6.2 million, this implies an average ADME/PK cost of approximately $0.6 million per candidate compound.

In practice, full DMPK preclinical packages (including method development, plasma stability, protein binding, and multi-species PK studies) typically cost between $150,000 and $235,000. A single in vivo ADME study—such as a radiolabelled mass balance experiment in one animal model—is typically priced in the low tens of thousands range per species. These figures apply both to the US and Europe, where pharmaceutical companies often outsource to established CROs for higher regulatory compliance and data quality. It is therefore clear that ADME evaluation is not a feasible tool for such products neither technically, nor economically. In addition, as already mentioned in the background art discussion, several drugs are reported to show relevant effects even after their excretion, therefore ADME, for this kind of drugs, misses essential information, i.e., it does not inform the developers and users of the therapeutic or beneficial product's latency.

On the contrary, the method of the present invention, provides a relevant information, i.e., the product's latency that goes beyond the analysed product's excretion time; is advantageously suitable for all therapeutic products, including natural matrices based products; and provides a functional and holistic measurement of the biological response to the product under examination independent of chemical markers quantification (being therefore independent from the batch-to-batch difference typical of theses products); allowing the identification of both transient and persistent biological effect (persistent effects are generally indicated by the persistence of transcriptomic perturbation in one or more organ sample when the transcriptomic perturbation induced by the product is resolved in most the other organ samples); and of safety-related molecular perturbations, and provides an objective characterisation of pharmacokinetic and pharmacodynamic profiles in natural matrices based products.

Summarising, the present invention is strongly advantageous over conventional ADME for many reasons. The present invention allows to monitor the organism's actual response rather than tracking each individual molecule. The method of the invention also allows temporal evidence of compound-host interplay, useful even when individual natural matrices-based therapeutic or beneficial product's components cannot be measured in plasma and the identification of persistent effects, therefore if gene expression remained altered beyond the expected time of action, it would be promptly observed, even in the absence of any “chemical trace” of the compound.

The method of the invention also provides an improved safety assessment, a gradual return to baseline suggests a transient effect, with no evidence of chronic molecular-level perturbations and a batch-to-batch robustness since, by detecting the extract's “functional” effect in vivo, transcriptomics reduces the impact of chemical variability among different lots.

Moreover, the present invention allows a stronger risk management: evaluating the actual biological (transcriptomic) response provides a more reliable parameter for assessing safety and efficacy, especially where lot-to-lot variations exist.

The invention provides a method for functionally characterising the onset, duration and resolution of the biological effects of a therapeutic or beneficial product following its administration to a subject, the method comprising:
(a) extracting RNA from biological samples of different tissues obtained from living organisms at multiple time points following administration of said natural matrices-based product, hereinafter treated sample(s), and from corresponding biological samples obtained at the same time points from a sham control living organism (i.e. administered following the same administration routes, amounts etc. used for the tested product, with a suitable placebo or vehicle), hereinafter sham control sample(s);
(b) performing transcriptomic analysis comprising gene expression profiling on the extracted RNA from each of said treated and sham control samples; and identifying, for each treated sample at each time point, transcriptomic perturbations in terms of significantly Differentially Expressed Genes (DEGs) relative to the corresponding sham control sample;
(c) defining the onset, duration, persistence, and resolution in time of said transcriptomic perturbations for each of said different tissues based on each transcriptomic perturbation identified in b) for samples of the same tissue;
wherein the method provides onset, duration, and resolution profiles of the biological effects of said product independent of direct quantification of individual chemical constituents thereof.

In other words, method of the invention allows to functionally characterise (i.e., understand the temporal dynamics of) the biological effects of a therapeutic or beneficial natural matrices-based product (complex, multi-component)-after it's been administered to a living subject by analysing RNA expression profiles (transcriptomics) from tissue samples over time, comparing samples from treated and sham control groups, and drawing conclusions about the onset, duration, and resolution of effects based on transcriptomic changes, rather than directly measuring specific chemical components of the product

Controls in the present invention are hence from sham-administered organisms sampled at the same times of the product-administered organisms. The living organisms are animals, preferably mammals commonly used in pre-clinical studies, excluding humans.

It is clear that sham and product treated animals as well as untreated control animals, are animals of the same species with the same genotype.

According to the method of the invention, the suitable control groups, for observing transcriptomic perturbation induced by a product administration, are sham-treated controls and not untreated controls. The present inventors found that confronting treated and untreated would display confounding effects (see FIG. 11) due to transcriptomic perturbations caused by circadian variations and/or treatment-related stress (e.g., injections), therefore, transcriptomic perturbation analysis, in order to provide more reliable and “background free” data, has to be performed confronting product treated and sham control treated samples of the same tissues collected at the same time points.

According to the method of the invention, said biological samples are samples from different tissues (multiple tissues) which are selected among the most representative of the whole subject response to the product's administration, in one non-limiting embodiment of the invention said tissues can be selected from one or more of blood, liver, kidney, brain, hypothalamus, lung, heart, spleen, testis, stomach, intestine, gallbladder, pancreas, various glands, ovaries, muscle tissues (by way of example, injection site when the administration is made by intramuscular injection). In a preferred embodiment said tissues will comprise at least blood, liver, kidney, brain, lung, and heart tissues.

Depending on the tested product putative therapeutic or beneficial effect and on the classes of patients for which the product would is intended, the skilled person can readily select the most appropriate representative tissues for carrying out the method of the invention. The product tested in the examples and reported in the figures is an anticancer product (also herein defined as EpigenAU/11, or product A). When reference is Clariom™ S Pico Assay, Mouse (Applied Biosystems, ThermoFisher Scientific) and the gene set thereof, the gene set is the set annotated on Jul. 30, 2018, by the manufacturer.

It is therefore clear that the method of the invention, provides relevant information on the analysed product's biological effects, that can embrace local effects in tissues from a large number of organs and/or systems of the treated subjects and therefore is able to provide much more exhaustive profile, “organism-level”, of the product's biological effects in time.

The samples are preferably samples treated, before storage, with tissue and RNA stabilisation reagents that preserve RNA integrity. Any suitable commercial product such as RNAlater® by Thermo Fisher Scientific, NA/RNA Shield™ (Zymo Research), Allprotect Tissue Reagent (QIAGEN), RNAprotect® Tissue Reagent (QIAGEN) and the like can be used, following the manufacturer's instructions.

According to the invention said multiple time points are multiple post-administration time points to track temporal effects, comprise TO, representing the time immediately after the administration of the product under examination and at least one, preferably at least two, even more preferably at least three or more different Tn, wherein n represents the hours after the administration of said product and is ≥1, preferably >2 and wherein one of said Tn is ≥to said resolution time point. Preferably, according to the invention, said Tn time points comprise T2 and T48. According to a non limited example, said time Tn points comprise at least three of T2, T6, T18, T24 and T48.

When the resolution time, i.e., the time at which significant transcriptomic perturbation is no longer detectable, is after 48 hours from TO, Tn will comprise a time point where n is >than 48, e.g., 72 or others.

As stated above, according to the invention, the samples are collected from two groups of animals: treated animals, i.e., animals treated with the product under examination and sham control animals, i.e., sham-treated animals which are animals treated exactly as the treated group, but the product administered is, by way of example, a placebo selected depending on the administration route and formulation of the product under examination. For each group of animals, the tissue samples are collected from each organ or tissue at the same time points and the transcriptomic comparison is made product-treated sample vs. the corresponding sham control sample at each time point.

In case T48 proves not sufficient for the resolution of the transcriptomic perturbations induced by the administration of the examined product, the method of the invention can be repeated with additional time points wherein n is >48. When the transcriptomic perturbation is resolved in most tested organs or tissues, the persistence of perturbation in one organ is in principle indicative of a persistent effect of the tested product in said organ, or even of a damage induced by the tested product in said organ.

Hence, contrary to ADME, the method of the invention, not only provides a holistic frame of the effects of a therapeutic or beneficial product, but it also allows to identify potential harmful effects of a tested product in specific organs or tissues. RNA can be extracted from the samples through any commonly known method or using any suitable commercially available kit.

Transcriptomic profiling can be carried out according to procedures commonly used in the art, such as RNA sequencing or hybridization to pre-designed gene expression microarrays. By way of example, cDNA can be obtained from the extracted RNA (total RNA can be extracted with any suitable commercial kit) and sequenced or the cDNA can be labelled and hybridised on commercial or tailored microarrays with pre-designed probes, or the profiling can be performed by directly hybridising the target RNA to fluorescent barcodes such as NanoString nCounter, or by qPCR-Based Arrays (e.g., TaqMan, Biomark Fluidigm) etc.

In one aspect of the invention, transcriptomic data are processed to ensure accuracy and comparability across samples. Generally, the data processing involves normalization to correct for technical variability and background noise. Preferably, normalization methods such as Signal Space Transformation-Robust Multiarray employed (https://assets.thermofisher.com/TFS-Average (SST-RMA) are Assets/GSD/Reference-Materials/white-paper-microarray %20-normalization-using-sst-with-probe-guanine-cytosine-count-correction.pdf). However, alternative normalization techniques known in the art, such as quantile normalization or cyclic loess normalization, may also be used depending on the platform and experimental setup. A suitable tool for such processing is, by way of example, Transcriptomic Analysis Console Software (ThermoFisher Scientific), which applies SST-RMA normalization, alternatively, a person skilled in the art may utilize Affymetrix Power Tools (APT), R/Bioconductor environments (e.g., ‘oligo’ or ‘affy’ packages), custom pre-processing pipelines replicating SST adjustments followed by RMA normalization, or other equivalent software capable of performing signal space transformation correction coupled with robust multiarray averaging.

Following normalization, Differential Expression (DE) analysis is performed to identify genes exhibiting statistically significant changes in expression between groups. In a preferred embodiment, DE analysis is conducted using linear models, such as those implemented in the Limma package from Bioconductor. Other suitable methods, including but not limited to DESeq2 or edgeR, may alternatively be utilized. The analysis involves estimating whether differences in gene expression between treatment groups (e.g., treated versus sham or untreated control) are statistically significant using linear models specifically designed for microarrays (Phipson, B, Lee, S, Majewski, IJ, Alexander, W S, and Smyth, GK (2016). Robust hyperparameter estimation protects against hypervariable genes and improves power to detect differential expression. Annals of Applied Statistics 10(2), 946-963). To control for the False Discovery Rate (FDR), the risk of false positives due to multiple testing, statistical adjustments are also applied. Preferably, the well-known Benjamini-Hochberg (BH) procedure is utilized (Benjamini, Yoav & Hochberg, Yosef. (1995). Controlling The False Discovery Rate-A Practical And Powerful Approach To Multiple Testing. J. Royal Statist. Soc., Series B. 57. 289-300. 10.2307/2346101), although other correction methods like the Bonferroni correction may also be applied. Genes are considered differentially expressed when their adjusted p-value falls below a predetermined threshold, typically 0.05, indicating strong evidence of genuine differential expression.

According to a non limiting example of the invention, as stated above, when testing significance for many genes (thousands of hypotheses at once), in order to avoid false positives, it is important to control for multiple testing. This can be done e.g., with the Limma package (“Linear Models for Microarray Data”) which is a widely used biostatistics software package and uses BH to control for the FDR (False Discovery Rate) (3), which is the expected proportion of false positives among the genes declared as “significant”. The procedure begins by collecting all the p-values from the individual tests and sorting them in increasing order. Each p-value is assigned a rank (i) based on its position in the sorted list, with the smallest p-value given rank 1, the second smallest rank 2, and so on up to the total number of tests (m). The FDR threshold is set to 0.05 and for each ranked p-value, you calculate a corresponding threshold value by multiplying the rank by the FDR threshold and dividing by the total number of tests.

According to the invention, genes are considered differentially expressed (DEGs) if their adjusted p-value is below 0.05. Lower values correspond to a lower expected proportion of false discoveries among the genes declared significant, reflecting stronger evidence of genuine differential expression.

$${p}_{( i )} \leq \frac{i}{m} \times FDR$$

In addition, the Molecular Degree of Perturbation (MDP) analysis can be employed to assess the global transcriptomic perturbation at the sample level. MDP analysis is based on calculating Z-scores for each gene, reflecting the deviation from the median expression of a reference (preferably untreated control samples). Alternative statistical measures such as t-scores or fold-change thresholds could also be considered for similar analyses. Only genes with significant deviations, i.e., with Z-scores absolute value≥2, are used to compute a perturbation score for each sample. The perturbation score represents the average magnitude of significant gene expression deviations, thereby quantifying the extent of transcriptomic disturbance.

Based on the DEG profiles over time, the method hence allows to determine the onset (which is when transcriptomic perturbations first appear after treatment), the duration/persistence (how long said perturbations last) and the resolution (return to baseline, end of perturbations) for each tissue across time points for each product tested.

As the method is based on the profiling of effects of the whole product, it is independent from its chemical composition as no specific chemical markers are needed.

As clear from the examples, the method of the invention provides consistent results for each tissue analysed. It is clear to the skilled person that the 48 hours time frame for product's effects clearance observed in the examples is related to the specific product tested, and that the time for the resolution of the transcriptomic perturbations may vary from product to product. In case different tissue samples show different resolution times of the transcriptomic perturbation induced by the administration of the product tested, when systemic resolution time is assessed, the resolution time corresponds to the first time period in which no more significant perturbations are observed when confronting the corresponding product treated and sham control samples for all the examined tissues.

In addition, the method can further comprise MDP analyses. DEGs analysis highlights specific genes with significant expression changes and provides the overall transcriptomic perturbation evolution over time, while MDP analysis provides an overall measure of transcriptomic shifts herein also indicated (see FIGS. 1-6 as “perturbation score”. This dual approach enables comprehensive characterization of the biological responses to treatments.

In an exemplary embodiment, transcriptomic data obtained from the treated biological samples as defined above, can be compared to sham-control samples alone or to sham-control samples and to baseline untreated samples. A DE analysis is performed by comparing product-treated samples (tissue/organ and time point specific as disclosed above) to their corresponding sham-treated samples at each time point, applying SST-RMA normalization, Limma-based modelling, and FDR correction.

In addition, normalized data can be used to calculate the Molecular Degree of Perturbation (MDP), expressed as a “perturbation score” of samples from both product- and sham-treated groups relative to an additional untreated control group. This analysis offers a global view of transcriptomic changes at the specific sample level. This method does not focus on individual genes but rather on the assessment of how globally “different” is this sample from a typical healthy control (untreated control sample) which is used as baseline. In the present specification, claims and drawings healthy sample and untreated control sample are considered synonyms.

MDP is derived from the concept of Molecular Distance to Health, which quantifies sample heterogeneity by evaluating how much a given sample's expression profile deviates from that of a healthy reference. The skilled person can use commonly available software such as the MDP package which calculates the perturbation score for each sample j by:
1. Computing a Z-score for each gene, a measure of how much the gene's expression in each sample differs from the typical value in healthy controls (expression values of each gene i subtracted the median of the same gene in control samples (Ci) divided by the variability of this gene in the control sample (Vi).
2. Only genes with significant deviations (absolute Z-score values≥2) are computed.
3. The perturbation score of the sample is the average of these significant deviations, representing how much the overall gene expression is “perturbed” compared to the untreated reference.

$${MDP}_{i} = \frac{1}{n} {\sum }_{i = 1} \vert \frac{{x}_{ij} - {μ}_{j} ( ref )}{{σ}_{j}^{( ref )}} \vert \qquad MDP Formula$$

wherein
MDPᵢ: Molecular degree of perturbation for subject i (sample perturbation/perturbation score)
η: Total number of genes used in the analysis
xᵢⱼ: Value of gene j in the subject i
μᵢ(ref): Mean of the gene j in the reference group (untreated controls)

$${σ}_{j}^{( ref )} :$$

Standard deviation of variable j in the reference group

Given that each gene j will be included in the analysis if, and only if, its absolute Z-score value is ≥2.

Therefore, the perturbations score for each sample i (MDPi) is calculated by doing an average of the Z-scores of the significant genes (absolute Z-score values≥2) for the respective sample i and then dividing by the total number of genes in the input data. The Z-scores are calculated taking the difference in expression level in sample i from the average of the gene in reference group divided by the corresponding standard deviation. Essentially, this represents the number of standard deviations (Prada-Medina, C. A., Fukutani, K. F., Pavan Kumar, N. et al. Systems Immunology of Diabetes-Tuberculosis Comorbidity Reveals Signatures of Disease Complications. Sci Rep 7, 1999 (2017). https://doi.org/10.1038/s41598-017-01767-4).

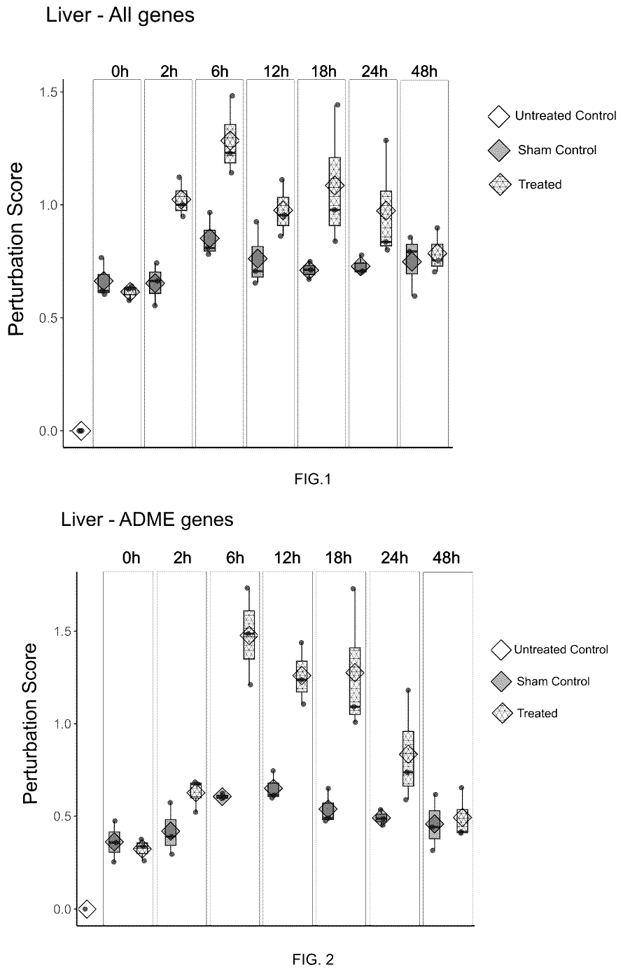

As clear from the examples hereinbelow and from the figures, both DEGs and MDP analyses revealed, for the product tested, that transcriptomic perturbation peaks shortly after the product's administration and subsides within 48 hours, indicating recovery trends over time. Additionally, in certain preferred embodiments, specific attention is directed toward analysing known ADME-related gene sets within liver and kidney tissues. In FIGS. 2 and 4, differentially expressed ADME genes are highlighted, and in MDP analysis, perturbation scores were computed using only ADME-related genes and provided a focused assessment on metabolic and excretory functions.

Furthermore, as indicated above, the method provides various ADME-like functional parameters such as:

| ADME | | | |
|---|---|---|---|
| CLASSIC | FUNCTIONAL | MEASURE | |
| PARAMETER | EQUIVALENT | PROVIDED | APPROACH |
| Cmax | Transcriptomic | Maximum intensity | Organ specific |
| | peak, Pmax | of the therapeutic | measurement |
| | | or beneficial | |
| | | product effect | |
| Tmax | Functional Tmax | Promptness of the | All-organs or |
| | (fTmax) | response to the | organ specific |
| | | administration of | measurement |
| | | the therapeutic or | |
| | | beneficial product | |
| AUC | AUEC (area | Overall volume of | All-organs or |
| | under the | the effect | organ specific |
| | effect curve) | (intensity × | measurement |
| | | duration) induced | |
| | | by the | |
| | | administration of | |
| | | the therapeutic or | |
| | | beneficial product | |
| MRT (Mean | Functional Mean | Mean time of the | All-organs |
| Residence | Residence Time | system (i.e., | |
| time) | (fMRT) | organism) | |
| | | perturbation | |
| | | following | |
| | | administration of | |
| | | the therapeutic or | |
| | | beneficial product | |
| Clearance | Resolution time | Reinstatement of | All-organs |
| | | the basal | |
| | | transcriptome (end | |
| | | of transcriptional | |
| | | perturbation) | |
| | | induced by the | |
| | | administration of | |
| | | the therapeutic or | |
| | | beneficial product | |

Hence, according to one embodiment the method herein disclosed further comprises one or more of:
a. providing the product's transcriptomic peak, Pmax, value for one or more organ representing the maximum intensity of the transcriptomic perturbation induced by the administration of said product in said organ,
b. providing the product's functional Tmax, fTmax, value for one or more organ representing the time point at which the maximum transcriptomic perturbation peak induced by the administration of said product in said organ is observed;
c. providing the systemic Area Under the Effect Curve, representing the area under the curve of the overall effect/perturbation induced by said product treatment over time
d. providing the systemic functional Mean Residence Time, fMRT, defining the average effect/perturbation time induced by said product treatment wherein fMRT=AUMC/AUEC, AUMC being the Area Under the first Moment Curve and AUEC being the Area Under the Effect Curve as defined in c.

The general MRT Formula is

$$MRT = AUMC / AUC$$

According to the invention, the fMRT Formula is

$$fMRT = \frac{AUMC}{AUEC} = \frac{\int t \cdot E ( t ) dt}{\int E ( t ) dt}$$

wherein
fMRT: functional Mean Residence Time
AUMC: Area under the First Moment Curve
AUEC: Area under the Effect Curve
t: time (hours)
E(t): Effect/Perturbation (number of DEGs) at time t

According to the invention and to the state of the art, integrals can be calculated using the Trapezoidal Estimate, therefore the formula above can be extended to:

$$fMRT = \frac{AUMG}{AUEC} = \frac{{\sum }_{i = 1}^{n - 1}}{{\sum }_{i = 1}^{n - 1}} \frac{\frac{( {t}_{i + 1} - {t}_{i} )}{2} [ {t}_{i} {E}_{i} + {t}_{i + 1} {E}_{i + 1} ]}{\frac{( {t}_{i + 1} - {t}_{i} )}{2} [ {E}_{i} + {E}_{i + 1} ]}$$

wherein
fMRT: functional Mean Residence Time
AUMC: Area under the First Moment Curve
AUEC: Area under the Effect Curve
n: Total number of measured time points in the time series
i: Index of the time interval between two consecutive time points
t_i: Time at the i th sampling point (with ‘i-th’ indicating the ordinal position i within a sequence of sampling time points)
t_(i+1): Time at the (i+1)th sampling point (with ‘i-th’ indicating the ordinal position i within a sequence of sampling time points)
E_i: Transcriptomic perturbation value at time t_i
E_(i+1): Transcriptomic perturbation value at time t_(i+1)

The Mean Residence Time is in general defined as the ratio AUMC/AUC.

In the present invention, AUC corresponds to its functional equivalent Area Under the Effect Curve (AUEC).

The trapezoidal rule can be used to estimate the total area under each curve by dividing it into trapezoids, and it is an accurate process for calculating integrals (Bolton, W. (2000). Mathematics for Engineering (2nd ed.). Routledge. https://doi.org/10.4324/9780080939292).

Specifically, AUMC is calculated as the area under the curve where the x-axis represents time, and the y-axis represents time multiplied by the Effect/Perturbation. In contrast, the AUEC is the area under the curve where the x-axis is also the time, but the y-axis represents only the Effect/Perturbation.

In classic pharmacokinetics, the MRT represents the average time a molecule stays in the body. As already stated, the method of the invention does not rely on parameters linked to molecules or concentrations of molecules, therefore the parameters provided herein are computed on the average effect/perturbation time of the analysed treatment.

The therapeutic or beneficial product can be any therapeutic or beneficial product, in a preferred embodiment, the product consists or comprises natural matrices, i.e., matrices obtained from natural sources, such as extracts from plants or animal tissues, mineral matrices produced by plants or animals (e.g., coral skeletons, eggshells and the like), fractions of said extracts and the like. In particular, extracts that do not alter the original components of the natural sources used, such as aqueous extracts are preferred.

Non limiting examples of plant matrices comprise plant-derived matrices such as officinal plants or plant parts extracts such as aloe vera gel, turmeric root extract, ginseng root extract, liquorice root extract, moringa leaf powder, Echinacea purpurea extract, neem oil, Arnica montana extract, and grape seed extract and the like; plant essential oils like tea tree, lavender, eucalyptus, frankincense and other essential oils obtained from officinal plants; plant fibres and scaffolds; resins and gums including myrrh, frankincense resin, acacia gum, and mastic gum; fungal matrices such as mushroom mycelium, reishi extract, cordyceps extract, and chaga extract; marine-derived materials such as fish collagen, krill oil, whole sea cucumber extract, brown seaweed extract, extracts or parts from algae, and nacre (mother of pearl) powder; animal-derived materials such as bovine or porcine collagen, placental extract, lanolin from sheep wool, keratin from feathers or hair, gelatine from animal bones, egg yolk or egg shell membrane extract, and colostrum; milk-derived bioactives including whole casein or whey protein concentrates and colostral fractions; insect-derived substances such as bee venom, propolis, royal jelly, silk fibroin from silkworm cocoons, and chitosan from insect or crustacean shells; microbial products like bacterial cellulose from Acetobacter, probiotics in whole-cell form, fermented plant extracts, and exopolysaccharide-rich microbial matrices; natural clays and mineral matrices enriched with bioactive plant or microbial components, such as montmorillonite clay loaded with herbal extracts.

In the meaning of the invention, these materials are typically used in their complex, multi-component form.

A specific non limiting example of natural matrices based therapeutic product is the EpigenAU/11 product disclosed in the examples. As clear from the present description and examples, the method of the invention offers the following significant advantages over conventional ADME-based strategies:

It is suitable for natural matrices-based products as it is independent from chemical markers.

It provides a functional and holistic measurement of biological response, independent of chemical marker quantification as it effectively bridges the gap between the measurable presence of individual molecular constituents and the persistence of pharmacological or biological effects.

Indeed, by capturing dynamic changes in gene expression over time, it enables the detection and characterization of functionally relevant biological responses even when the original matrix components are no longer detectable in plasma or tissues. In addition, by analysing the entire protein-coding transcriptome and not only ADME-related genes the method of the invention ensures that any relevant biological signal is captured, whether: adaptive, beneficial, toxicological, or arising from indirect or systemic mechanisms of action.

It provides an accurate and time-resolved mapping of the onset, peak, and resolution phases of the product's pharmacodynamic activity, indeed, the analysis of gene expression patterns according to the invention provides a dynamic and temporal profile of biological activity, enabling the detection of pharmacodynamic effects even when the original chemical constituents are no longer detectable in plasma or tissues. The method provides a detailed and temporally resolved map of the product's activity across multiple organs, highlighting the sequential occurrence of metabolic (hepatic), excretory (renal), and systemic responses.

The temporal resolution enables a clear distinction between early and late biological effects, supporting a comprehensive understanding of the tested product's pharmacokinetic and pharmacodynamic profile.

It enables detection both transient and persistent biological effects, indeed, any long-lasting molecular alterations induced by the product tested would be readily identified, allowing for an objective assessment of pharmacodynamic persistence or delayed toxicological signals.

It provides identification of safety-related molecular perturbations, as the progressive return of transcriptomic signatures to baseline values over time represents a robust and reliable indicator of biological recovery and the absence of chronic molecular perturbations.

By focusing on the functional biological outcome rather than the chemical composition of the extract, this method mitigates the variability associated with differences in product composition between production batches

As discussed, and shown above and in the examples, the method of the invention provides ADME-like parameters and therefore it applicable to Enhanced Risk Management and Regulatory requirements. The analysis of genome-wide gene expression profiles provides a biologically relevant parameters for evaluating safety, efficacy, and consistency of multicomponent botanical products, improving the robustness of preclinical and regulatory dossiers.

Ultimately, the present invention represents a significant methodological advancement in the pharmacokinetic and pharmacodynamic assessment of multicomponent natural products.

By shifting the analytical focus from static chemical quantification to dynamic biological response profiling, the proposed approach provides a more realistic, comprehensive, and biologically relevant alternative to conventional ADME methodologies, with direct applicability in preclinical development, safety assessment, efficacy profiling, regulatory dossiers, and product standardization strategies for botanical and natural matrices-based therapeutics.

This method allows for an unprecedented functional profiling of natural matrices, overcoming the intrinsic limitations of traditional ADME approaches but provides ADME-like information and is therefore applicable across multiple therapeutic and regulatory contexts.

The overall advantages enable for an objective characterization of pharmacokinetic and pharmacodynamic profiles of products comprising or consisting of plant natural matrices.

In any part of the present description and claims the term comprising can be substituted by the term consisting of.

In any part of the description, when an internet address or URL is provided, it relates to the information retrieved from said internet address available at the filing date of the present application, i.e., at the latest version of the content at said internet address at the filing date of the present application.

In any part of the description or in the claims, when reference is made to commercial products, the symbols TM (™) or © are considered as implicit and can be added to each of said products at any time.

The following examples illustrate, without limiting, the foregoing description, such examples are in no way to be considered as a limitation of the previous description and the subsequent claims, further, the examples below report all the studies performed on the product of the invention and support all the subject-matter claimed.

### EXAMPLES

### 1. Composition of the Tested Product

Product A, herein, also EpigenAU/11 for the treatment of cancer.
36.05% in weight of freeze-dried component 1
63.06% in weight of freeze-dried component 2
0.89% in weight of freeze-dried component 3 for a total of 100% w/w of product A.

### Component 1

Laurus nobilis leaves 25% w/w
Withania somnifera roots 25% w/w
Filipendula vulgaris leaves and flowers 25% w/w
Brassica oleracea L. Botrytis cymosa seeds 25% w/w
For a total of 100% w/w, coextracted in water

### Component 2

Cynara scolymus L. leaves 14.30% w/w
Curcuma longa L. roots 42.85% w/w
Tanacetum parthenium L. flowers 42.85% w/w
For a total of 100% w/w, coextracted in water

### Component 3

Agave sisalana leaves freeze-dried extract.

All animal manipulations were carried out according to the Directive 2010/63/EU of the European Parliament and the European Union Council (22 Sep. 2010) on the protection of animals used for scientific purposes. The ethical policy of the University of Florence complies with the Guide for the Care and Use of Laboratory Animals of the US National Institutes of Health (NIH Publication No. 85-23, revised 1996; University of Florence assurance number: A5278-01). Formal approval to conduct the experiments described was obtained from the Animal Subjects Review Board of the University of Florence. Experiments involving animals have been conducted according to the ARRIVE guidelines. All efforts were made to minimize animal suffering and reduce the number of animals used.

### 2 Exemplary Application of the Invention: EpigenAU/11 Natural Matrices-Based Product

As a non-limiting example of application, the proposed method was applied to the EpigenAU/11, already patented, in order to characterize its temporal transcriptomic impact in vivo.

### 2.1 Collected Samples

A total of 45 immunodeficient nude mice (nu/nu) were used. The animals were maintained under standard barrier facility conditions (temperature 22±2° C.; relative humidity 50-60%; 12/12-hour light/dark cycle) with sterile food and water provided ad libitum.

Experimental groups included:
Baseline group (0 h, no injection): 3 mice.
Treatment groups: Subcutaneous injection of EpigenAU/11 (50 mg/mL), 400 μL per animal.
Sham control groups: Subcutaneous injection of physiological saline (vehicle), 400 μL per animal.
Tissue samples were collected at the following predefined time points post-injection: 0 h, 2 h, 6 h, 12 h, 18 h, 24 h, 48 h.

At each time point, 6 animals were sacrificed (3 treated with EpigenAU/11 and 3 with vehicle).

The collected biological samples included:
Blood (500 μL, stabilized in RNAprotect tubes)
Injection site tissue
Brain
Liver
Kidney
Lung
Heart
Muscle (from the injection site area)

Samples were processed and preserved in RNAlater at 4° C. for 24 hours and subsequently stored at −80° C. for RNA extraction.

### 3. RNA Extraction and Transcriptomic Profiling

Tissue homogenization was performed in RLT buffer (Qiagen) with β-mercaptoethanol and DX reagent.

Total RNA was isolated using the QIAsymphony RNA Kit and QIAsymphony SP instrument.

RNA integrity was verified by Agilent 2100 Bioanalyzer and quantified by spectrophotometry.

Whole-transcriptome analysis was conducted using Clariom™ S Pico Assay, Mouse (Thermo Fisher Scientific) as defined in the figures section above on the GeneTitan® MC Instrument, followed by SST-RMA normalization.

### 4. Data Processing and Analysis

Two analytical strategies were employed:

### Molecular Degree of Perturbation (MDP)

Global quantification of transcriptomic deviation compared to untreated controls.
Captures subtle and widespread gene expression changes across the entire transcriptome.

### Differential Expression Analysis (DE) or (DEA)

Identification of specific genes showing statistically significant changes between product-treated and vehicle-treated animals at each time point thereby obtaining DEGs (differentially expressed genes).

### 4.1 De Analysis:

Transcriptomic data were processed using the Transcriptomic Analysis Console Software (ThermoFisher Scientific), which applies SST-RMA normalization—a method developed by this software to adjust for technical variability and background noise, ensuring that gene expression values are comparable across all samples (https://assets.thermofisher.com/TFS-Assets/GSD/Reference-Materials/white-paper-microarray%20-normalization-using-sst-with-probe-guanine-cytosine-count-correction.pdf). The Transcriptomic Analysis Console software also performs Differential Expression (DE) analysis (based on the linear models from Limma Bioconductor package). This analysis estimates whether differences in gene expression between groups (e.g., EpigenAU/11 vs Physiological Solution or Physiological Solution vs Untreated) are statistically significant using linear models specifically designed for microarray platforms (Phipson, B, Lee, S, Majewski, IJ, Alexander, W S, and Smyth, GK (2016). Robust hyperparameter estimation protects against hypervariable genes and improves power to detect differential expression. Annals of Applied Statistics 10(2), 946-963).

When testing significance for many genes (thousands of hypotheses at once), there is a risk of getting some false positives just by chance. To avoid this issue, it is important to adjust for multiple testing, which is done by Limma package controlling the FDR (False Discovery Rate) with the BH method (Benjamini, Yoav & Hochberg, Yosef. (1995). Controlling The False Discovery Rate-A Practical And Powerful Approach To Multiple Testing. J. Royal Statist. Soc., Series B. 57. 289-300. 10.2307/2346101), which is the expected proportion of false positives among the genes you declare as “significant”. The procedure begins by collecting all the p-values from the individual tests and sorting them in increasing order. Each p-value is assigned a rank (i) based on its position in the sorted list, with the smallest p-value given rank 1, the second smallest rank 2, and so on up to the total number of tests (m). The False Discovery Rate (FDR) threshold is set to 0.05 and for each ranked p-value, you calculate a corresponding threshold value by multiplying the rank by the FDR threshold and dividing by the total number of tests.

$${p}_{( i )} \leq \frac{i}{m} \times FDR$$

Genes were considered differentially expressed (DEGs) if their adjusted p-value was below 0.05. Lower values correspond to a lower expected proportion of false discoveries among the genes declared significant, reflecting stronger evidence of genuine differential expression.

### 4.2 MDP

Normalized data was used to calculate the Molecular Degree of Perturbation (MDP) of samples from both EpigenAU/11—and Sham control groups relative to the untreated control group. This analysis offers a global view of transcriptomic changes at the sample level. This method does not focus on individual genes but rather asks: How globally “different” is this sample from a typical healthy control?

MDP is derived from the concept of Molecular Distance to Health, which quantifies sample heterogeneity by evaluating how much a given sample's expression profile deviates from that of a healthy reference. The MDP package calculates the perturbation score for each sample j by:
1. Computing a Z-score for each gene, a measure of how much the gene's expression in each sample differs from the typical value in healthy controls (expression values of each gene i subtracted the median of the same gene in control samples (Ci) divided by the variability of this gene in the control sample (Vi).
2. Only genes with significant deviations (Z-scores absolute value≥2) are computed.
3. The perturbation score of the sample is the average of these significant deviations, representing how much the overall gene expression is “perturbed” compared to the reference.

$${MDP}_{i} = \frac{1}{n} {\sum }_{j = 1} \vert \frac{{x}_{ij} - {μ}_{j} ( ref )}{{σ}_{j}^{( ref )}} \vert \qquad MDP Formula$$

MDPᵢ: Molecular degree of perturbation for subject i (sample perturbation)
n: Total number of genes used in the analysis
x_{if}: Value of gene j in the subject i
μⱼ(ref): Mean of the gene j in the reference group (untreated controls)

$${σ}_{j}^{( ref )} :$$

Standard deviation of variable j in the reference group

Given that each gene j will be included in the analysis if, and only if, its absolute Z-score is higher than 2.

Therefore, each Sample Perturbation (MDPi) is calculated by doing an average of the Z-scores of the significant genes (Z-scores with absolute value≥2) for the respective sample i and then dividing by the total number of genes in the input data. The Z-scores are calculated taking the difference in expression level in sample i from the average of the gene in reference group divided by the corresponding standard deviation. Essentially, this represents the number of standard deviations from reference (1).

The dual approach ensures both the detection of systemic transcriptomic shifts and the identification of key driver genes involved in the biological response.

Importantly, sham control-treated samples also presented transcriptomic perturbation compared with untreated control samples. This reflects not only the fluctuations of gene expression throughout the day, including the circadian rhythm, but also the other effects such as the stress caused by the injection (FIG. 11). For this reason, the DE analysis was conducted by comparing EpigenAU/11-treated groups at each time point to their corresponding groups treated with the physiological solution. This approach was designed to minimize the confounding effects of gene expression fluctuations occurring throughout the day.

The Molecular Degree of Perturbation (MDP) approach assesses at sample level the overall transcriptomic perturbation of each sample by quantifying deviations across the entire gene expression profile to the median values of the untreated control group, without performing statistical tests on individual genes. On the other hand, Differential Expression (DE) Analysis applies statistical testing to identify specific genes with significant changes in expression between conditions. These two methods are complementary. MDP captures subtle, global shifts in the transcriptome for all the genes or a specific subset comparing each sample to the untreated control group at time point 0. DE analysis highlights distinct genes that may be key drivers of the biological response, looking for group differences between EpigenAU/11 treatment against sham-treatment at each time point. For this reason, both methods are used and do not always yield the same results. Importantly, both suggest that the peak of the perturbation caused by EpigenAU/11 in the different organs decreases by 48 hours post-administration.

For the Liver and the Kidneys, both approaches were given an extra focus on the genes possibly related to the ADME processes based on datasets described for each organ (Hu D G, Mackenzie P I, Nair P C, Mckinnon R A, Meech R. The Expression Profiles of ADME Genes in Human Cancers and Their Associations with Clinical Outcomes. Cancers (Basel). 2020 Nov. 13; 12(11): 3369. doi: 10.3390/cancers12113369. PMID: 33202946; PMCID: PMC7697355 for liver, and Schlosser, P., Li, Y., Sekula, P. et al. Genetic studies of urinary metabolites illuminate mechanisms of detoxification and excretion in humans. Nat Genet 52, 167-176 (2020). https://doi.org/10.1038/s41588-019-0567-8 for kidneys). In the DE analysis the number of DEGs that were part of the ADME specific list of these organs were highlighted. For the MDP, the whole analysis was performed using only these genes of interest.

### 5. Results and Discussion

### 5.1 Overview of the Observed Biological Response

The application of organ-specific transcriptomic analysis to the EpigenAU/11 revealed a clear, time-dependent biological response across multiple organs in vivo. Two complementary analytical strategies were employed to evaluate the transcriptomic changes induced by the product:

Differential Expression Analysis (DEA), performed to identify statistically significant gene expression changes between EpigenAU/11-treated animals and appropriate sham controls. Following this analysis, the differentially expressed genes (DEGs) were further annotated to specifically highlight genes belonging to the ADME-related category, when relevant.

Molecular Degree of Perturbation (MDP), used to quantify the overall transcriptomic deviation of each sample from the untreated control considered as the reference physiological state, providing a global and integrated measure of gene expression perturbation.

This dual analytical framework allowed a comprehensive characterization of the biological response to EpigenAU/11 administration, enabling both a broad/extensive evaluation of the transcriptomic landscape and a targeted focus on metabolism-specific processes (ADME genes).

### 5.2 Liver: Metabolic Activation and Detoxification

The liver showed the strongest transcriptomic response following EpigenAU/11 administration.

A first increase in gene expression perturbation was observed approximately 2 hours post-administration, peaking at 6 hours, as demonstrated by MDP analysis (FIG. 1) and confirmed by DEA (FIG. 7).

This approach was also applied to a subset of the dataset, selecting only the genes related to the ADME (Absorption, Distribution, Metabolism and Excretion) processes, according to a gene set used in a previous work by Dong Gui Hu et al (Dong Gui Hu et al. The Expression Profiles of ADME Genes in Human Cancers and Their Associations with Clinical Outcomes Cancers 2020, 12(11), 3369).

When focusing specifically on ADME-related genes (FIG. 2), a similar temporal pattern was observed, but with less perturbation at 2 hours, consistent with the activation of hepatic metabolism and detoxification pathways. The differences between physiological solution and EpigenAU/11-treated became more evident.

Importantly, at 48 hours post-administration, both global and ADME-specific perturbation levels returned to values comparable to the vehicle-treated group (FIGS. 1 and 7), indicating complete resolution of the hepatic response.

### 5.3 Kidney: Excretory Dynamics and Biphasic Response

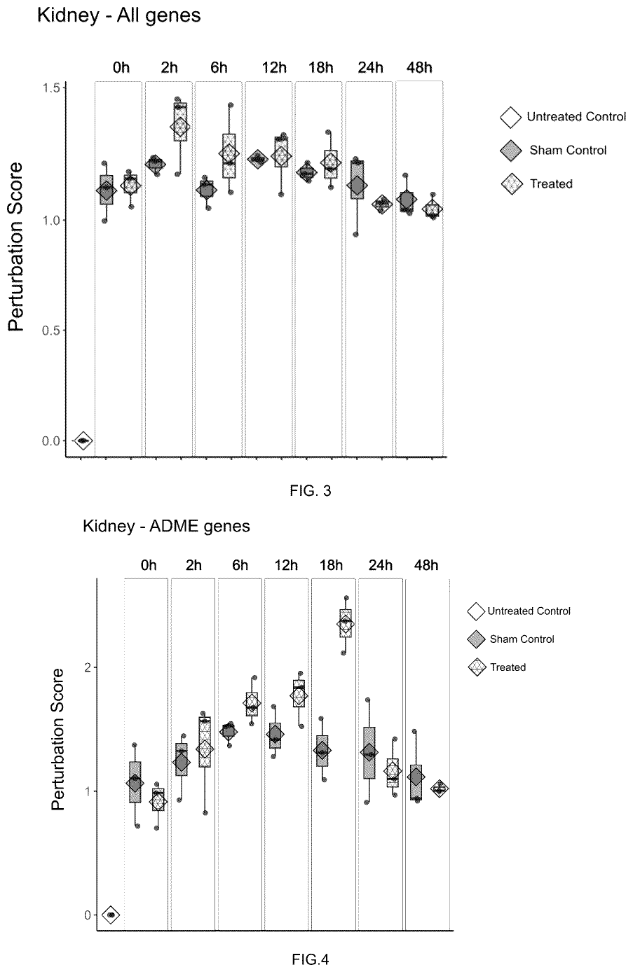

In the kidney, transcriptomic perturbation peaked initially at 2 hours post-administration when considering all genes (FIG. 3).

Again, MDP was used to assess both overall transcriptomic perturbation and specific perturbations linked to ADME processes. The gene set chosen for the kidney is described by a Genome-wide association study (GWAS) of metabolite concentrations (Schlosser, P., Li, Y., Sekula, P. et al. Genetic studies of urinary metabolites illuminate mechanisms of detoxification and excretion in humans. Nat Genet 52, 167-176 (2020). https://doi.org/10.1038/s41588-019-0567-8), 63 genes were selected (genes found by the GWAS and that are known to participate on metabolic-related pathways in general).

The MDP analysis of ADME-related genes (FIG. 4) revealed a distinct second peak of perturbation at approximately 18 hours post-administration, likely reflecting renal excretion processes.

DEA results corroborated this biphasic pattern, showing a first perturbation peak at 2 hours and a second at 18 hours (FIG. 8). By 48 hours post-administration, kidney transcriptomic profiles returned to baseline levels.

5.4 Blood: Minimal Perturbations with Transient Signals

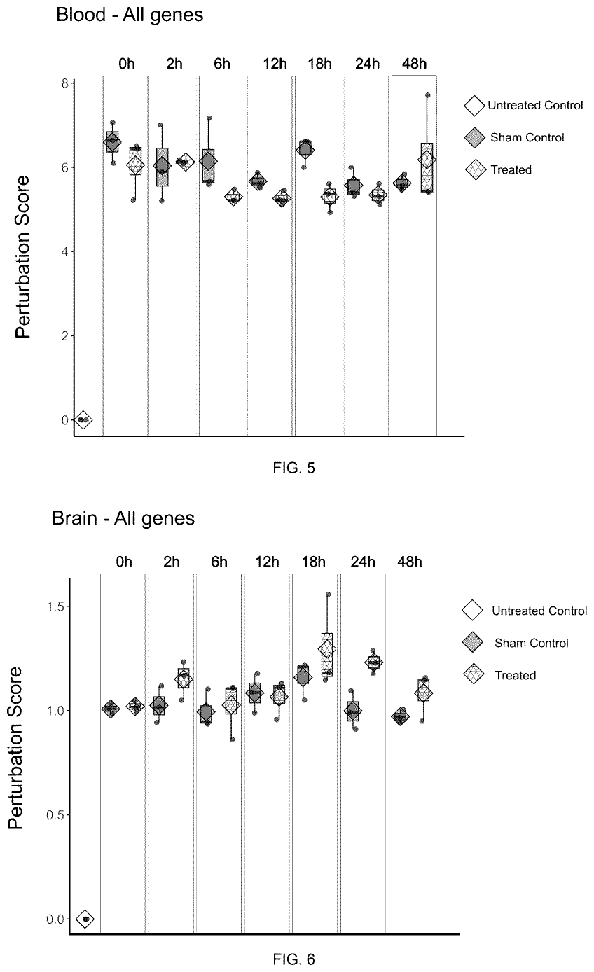

Blood samples exhibited overall low perturbation scores across the time course (FIG. 5).

However, two notable findings emerged:

At 18 hours post-administration, vehicle-treated samples showed a higher perturbation level than EpigenAU/11-treated samples—likely reflecting stress-related or circadian effects.

A minor outlier was detected at 48 hours in one EpigenAU/11-treated sample.

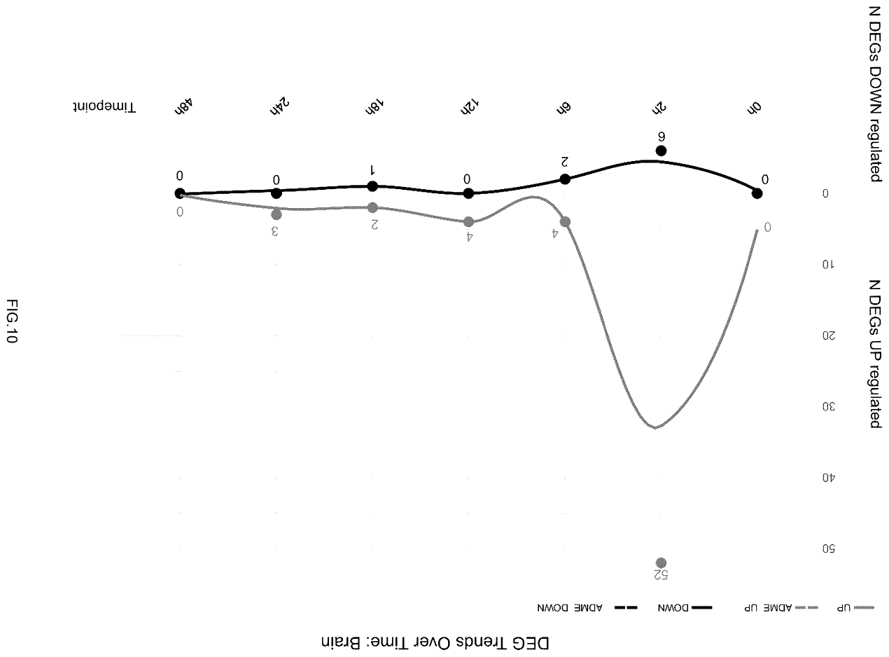

Despite these exceptions, DEA analysis revealed a significant number of differentially expressed genes (DEGs) at 18 hours (FIG. 9), suggesting dynamic transcriptomic changes in blood, although not directly attributable to EpigenAU/11 exposure alone.

### 5.5 Brain: Detection of Indirect and Systemic Effects on CNS

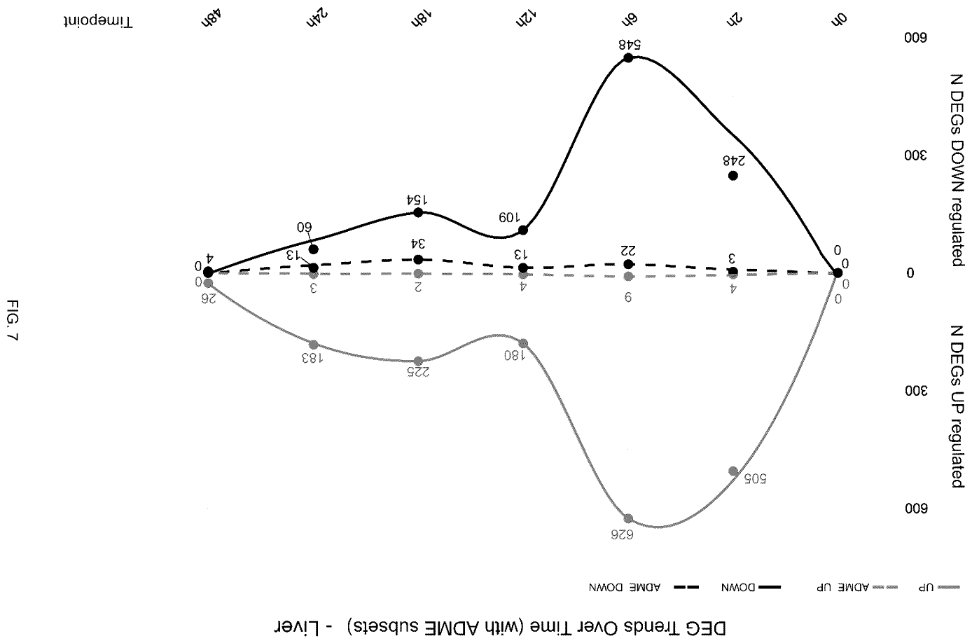

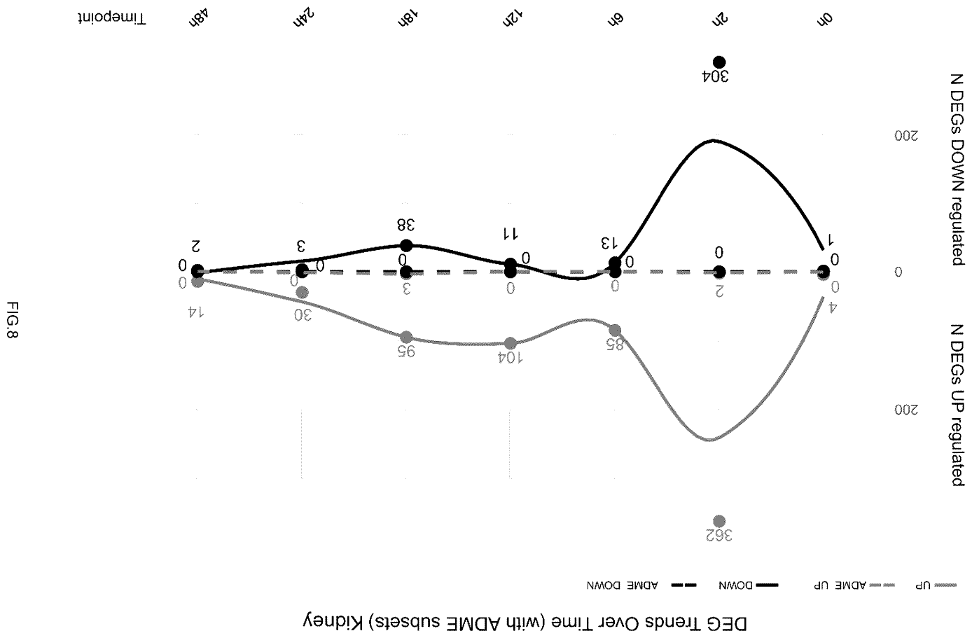

Transcriptomic perturbation in the brain was detectable approximately 2 hours post-administration of EpigenAU/11 (FIG. 6), in temporal alignment with the perturbation signals observed in peripheral metabolic organs such as the liver and kidney.

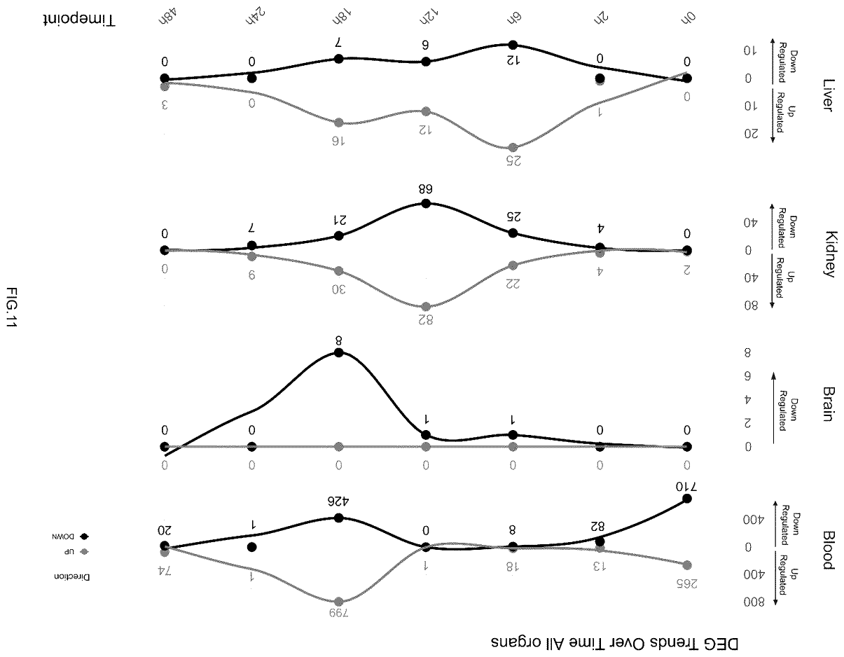

At subsequent time points (24 and 48 hours), MDP analysis continued to indicate a slight but measurable deviation from baseline gene expression in brain tissue, despite the absence of significant differentially expressed genes (DEGs) identified by DEA from 6 hours onward (FIG. 10).

This finding is of particular importance, as it demonstrates that the proposed transcriptomic analysis method is capable of capturing indirect, systemic, or secondary biological effects occurring in the brain—even in situations where the administered natural matrices based-product, or its detectable metabolites, may not directly reach or accumulate in the central nervous system (CNS).

Such capacity to detect CNS-relevant transcriptomic changes—arising from systemic modulation, peripheral signalling, or metabolite-driven pathways—represents a key advantage of the method, especially considering that conventional pharmacokinetic strategies typically fail to capture these indirect effects.

This ability to monitor subtle brain gene expression modulations, beyond direct compound penetration through the blood-brain barrier, provides valuable insights into:
the extended systemic impact of natural matrices-based products, potential CNS-related efficacy signals,
and the early detection of safety-relevant alterations in the brain transcriptome, even for compounds classically considered CNS-inert.

This feature is particularly advantageous for safety profiling, pharmacodynamic evaluation, and risk assessment of botanical products or xenobiotics that are not expected to directly act within the CNS compartment.

### 5.6 Resolution Phase Across all Organs

By 48 hours post-administration, transcriptomic profiles in all examined organs—including liver, kidney, blood, and brain—had returned to levels comparable to sham controls.

This resolution phase is consistent with the expected sequence of:
hepatic metabolic processing,
renal excretion, and
systemic recovery
typically associated with xenobiotic elimination.

The dual analytical strategy applied here—combining ADME-specific gene analysis with whole-transcriptome profiling—offers a uniquely detailed and comprehensive view of EpigenAU/11 metabolism and biological action.

### 5.7 Safety and Tolerability of EpigenAU/11

A key observation emerging from the present analysis is the absence of persistent or chronic molecular perturbations following EpigenAU/11 administration.

Specifically:

Gene expression profiles in both liver and kidney returned to baseline levels within 48 hours post-administration.

No lasting transcriptomic alterations were detected, suggesting that the PRODUCT was effectively processed and eliminated within a reasonable and predictable timeframe.

Under physiological conditions (healthy animal model), no molecular signatures indicative of chronic toxicity, stress response, or irreversible damage were observed. It is important to emphasize that the transcriptomic analysis was conducted across the entire protein-coding expression profile, without limiting the investigation to canonical ADME-related genes (such as cytochrome P450 enzymes, conjugating enzymes, or membrane transporters).

This comprehensive strategy ensures that any potential biological effect—whether metabolic, inflammatory, adaptive, or toxicological—could be detected and assessed.

By capturing both direct and indirect effects, including those potentially arising from metabolites or systemic modulation, this approach provides a uniquely broad and reliable assessment of safety and pharmacodynamic behaviour.

The organ-specific transcriptomic analysis applied to the administration of EpigenAU/11 clearly demonstrates the feasibility, robustness, and added value of this innovative approach in characterizing the pharmacokinetic and pharmacodynamic behaviour of complex botanical extracts.

This methodological strategy addresses several longstanding limitations inherent to traditional ADME-based analysis and provides a functionally relevant, biologically integrative framework applicable to the study of other multicomponent natural matrices-based products.

In particular, while the targeted ADME gene analysis provides clear information on the pharmacokinetic processing of the compound, the transcriptome approach captures broader systemic responses, indirect effects, and tissue-specific adaptations—providing a truly holistic evaluation of complex natural matrices-based products behaviour in vivo.

analysis.

### 6. ADME-Like Functional Parameters

Calculation of functional/equivalent ADME Parameters as defined above and in the related table was performed.

The functional parameters were calculated based on the extent of the biological response defined by the total number of differentially expressed genes (DEGs) observed when comparing treated samples to their corresponding sham control groups.

The table below shows the results of the DE analysis, indicating the number significant DEGs detected at each time point for each tissue sample and the overall (systemic) number of significant DEGs at each time point.

| Organ | 0 h | 2 h | 6 h | 12 h | 18 h | 24 h | 48 h |
|---|---|---|---|---|---|---|---|
| Blood | 469 | 0 | 49 | 180 | 801 | 5 | 89 |
| Brain | 0 | 58 | 6 | 4 | 3 | 3 | 0 |
| Kidneys | 5 | 666 | 98 | 115 | 133 | 33 | 16 |
| Liver | 0 | 753 | 1174 | 289 | 379 | 243 | 28 |
| Sum | 474 | 1477 | 1327 | 588 | 1316 | 284 | 133 |

To note, T0, which is few minutes after injection of EpigenAU/11, shows that at the blood level the mere administration procedure is likely to provoke transcriptomic perturbation almost immediately.

All calculations were conducted separately for each organ at each evaluated time point, thereby enabling organ-specific assessments of molecular perturbation. Additionally, a global systemic perturbation profile was derived by summing the number of DEGs across all evaluated organs at each individual time point, providing a holistic view of the treatment-induced biological response over time.

### 6.1 Pmax

The maximum perturbation (Pmax) for a given organ defines the maximum number of observed DEGs observed in the treated samples compared to the Sham control, regardless of whether the genes are upregulated or downregulated.

As evident from the table above, in the blood, the peak is of 801 DEGs, in the brain 58, 666 in the kidneys and 1174 DEGs in the Liver.

For the systemic effect, the Pmax is the overall maximum number of observed DEGs at a given time point and which is 1477.

6.2 fTmax

The functional time to maximum effect (fTmax) value for one or more organ is the time point at which the maximum transcriptomic perturbation peak induced by the administration of said product in said organ, the fTmax systemic value is the time point at which the highest number of DEGs is observed when all the organ specific DEGs are taken into account. As clear from the table above after administration of EpigenAU/11, the most pronounced perturbation (Pmax) in the liver, was characterized by 1174 DEGs and was observed 6 hours (fTmax=6 h); in the blood, kidneys and brain fTmax was at 18 hours, fTmax at the systemic level defines the time point at which the overall highest perturbation is observed, as clear from the table above, systemic fTmax was 2 hours.

### 6.3 AUEC

To evaluate the overall temporal perturbation profile, the Area Under the Effect Curve (AUEC), i.e., the area under the curve of the overall effect/perturbation induced by the product treatment over time was calculated by plotting the number of DEGs (y-axis) as a function of time (x-axis) and calculating the area under the resulting curve using the trapezoidal rule. The implementation was performed via the trapz( ) function from the pracma package in R. This method ensures numerical accuracy in approximating the area under the curve based on discrete time-series data.

The following formula was used for calculating systemic AUEC

$$AUEC = \int {E}_{( t )} dt$$

Wherein
AUEC: Area under the Effect Curve
t: time (hours)
E(t): Effect/Perturbation (number of DEGs) at time t

In the liver, for instance, the calculation would be as following (using the trapezoidal rule on described page 44):

$${AUC}_{Liver} = {\int }_{0}^{48 h} E ( t ) dt \approx {\sum }_{i = 0}^{n - 1} \frac{{E}_{i} + {E}_{i + 1}}{2} ( {t}_{i + 1} - {t}_{i} )$$

| interval | Eᵢ | Eᵢ₊₁ | Δt (h) | contribution |
|---|---|---|---|---|
| 0-2 h | 0 | 753 | 2 | $\begin{matrix}0 + 753 \\ 2\end{matrix} \times 2 = 753$ |
| 2-6 h | 753 | 1174 | 4 | $\frac{753 + 1174}{2} \times 4 = 3854$ |
| 6-12 h | 1174 | 289 | 6 | $\frac{1174 + 289}{2} \times 6 = 4389$ |
| 12-18 h | 289 | 379 | 6 | $\frac{289 + 379}{2} \times 6 = 2004$ |
| 18-24 h | 379 | 243 | 6 | $\frac{379 + 243}{2} \times 6 = 1866$ |
| 24-48 h | 243 | ↓ | 24 | $\frac{243 + 28}{2} \times 24 = 3252$ |

$$AUC Liver \approx 753 + 3854 + 4389 + 2004 + 1866 + 3252 = 16118$$

All calculations were made, mutatis mutandis, for the other organs.

The calculated systemic AUEC, representing the overall (all organs) effect size of the perturbation was of 28820. When the effect size was measured for single organs, the resulting value was of 16118 for the liver, 4668 for the kidneys, 7743 in the blood and 291 for the brain. Calculation of AUEC for distinct tissue samples can show relevant differences in the tissue-by-tissue effect size. By way of examples, the data above show that the effect size of EpigenAU/11 administration is significantly larger in the liver than in the brain.

6.4 fMRT

The Functional Mean Residence Time (fMRT), which defines the average effect/perturbation time induced by the product treatment fMRT was calculated as the ratio of the Area Under the Moment Curve (AUMC) to the AUEC. The AUMC was derived by calculating the area under the curve where the y-axis represents the mathematical product of the time point and the corresponding number of DEGs (i.e., time×effect), again using the trapz( ) function for trapezoidal approximation.

Although the calculation of this average perturbation time relates closely to a systemic effect, in the case of interest in a specific organ, it can also be calculated for the single organs. EpigenAU/11 administration resulted in organ-specific fMRTs of 12.72 hours in the liver, 10.86 hours in the kidneys, 19.89 hours in the blood and 7.63 hours in the brain). The systemic functional mean residence time was 14.29 hours for our product.

The formula used was

$$fMRT = \frac{AUMC}{AUEC} = \frac{\int t \cdot E ( t ) dt}{\int E ( t ) dt}$$

wherein
fMRT: functional Mean Residence Time
AUMC: Area under the First Moment Curve
AUEC: Area under the Effect Curve
t: time (hours)
E(t): Effect/Perturbation (number of DEGs) at time t

In the liver, for instance, the calculation would be as following:
I. Calculate the AUEC as displayed in page 60:

$${AUC}_{Liver} = {\int }_{0}^{48 h} E ( t ) dt \approx {\sum }_{i = 0}^{n - 1} \frac{{E}_{i} + {E}_{i + 1}}{2} ( {t}_{i + 1} - {t}_{i} )$$

$$AUC Liver \approx 753 + 3854 + 4389 + 2004 + 1866 + 3252 = 16118$$

II. Calculate the AUMC as the formula above:

tᵢEᵢ: 0, 1506, 7044, 3468, 6822, 5832, 1344

| interval (h) | tᵢEᵢ | tᵢ₊₁Eᵢ₊₁ | Δt | contribution to AUMC |
|---|---|---|---|---|
| 0-2 | 0 | 1 506 | 2 | $\frac{0 + 1506}{2} \times 2 = 1506$ |
| 2-6 | 1 506 | 7 044 | 4 | $\frac{1506 + 7044}{2} \times 4 = 17100$ |
| 6-12 | 7 044 | 3 468 | 6 | $\frac{7044 + 3468}{2} \times 6 = 31536$ |
| 12-18 | 3 468 | 6 822 | 6 | $\frac{3468 + 6822}{2} \times 6 = 30870$ |
| 18-24 | 6 822 | 5 832 | 6 | $\frac{6822 + 5832}{2} \times 6 = 37962$ |
| 24-48 | 5 832 | 13 ↓ | 24 | $\frac{5832 + 1344}{2} \times 24 = 86112$ |

$$AUMC Liver = 1506 + 17100 + 31536 + 30870 + 37962 + 861 \; 12 = 205086$$

$${fMRT}_{Liver} = \frac{AUMC}{AUEC} = \frac{205 \; 086}{16 \; 118} \approx 12.7 hours$$

All calculations were made, mutatis mutandis, for the other organs.

6.5 fTres

Organ specific functional resolution time fTres corresponds to the time point at which reinstatement of the basal transcriptome (end of transcriptional perturbation, defined as a transcriptomic perturbation≤5% of the Pmax for all tissue samples excluded blood wherein the end of transcriptomic perturbation is defined as ≤12% Pmax, preferably ≤5% Pmax) induced by the administration of the therapeutic or beneficial product. As can be expected, transcriptomic perturbation in blood samples is more unstable as blood stream is more susceptible to alterations also due to variations such as glycaemic peaks immediately after feeding, day-night alternations and the like. Also, in this case fTres can be organ specific or systemic. When considered under a systemic point of view, fTres is the first time point at which all organs tested reach organ specific a transcriptomic perturbation≤5% of the Pmax for excluded blood wherein the end of transcriptomic perturbation is defined as ≤12% Pmax, preferably ≤5% Pmax.
In the current study, fTres was 48 hours for all tissue samples tested as well as for the systemic value.

The table below summarises the calculated ADME-like functional parameter obtained with the method of the invention following the administration of EpigenA1/11.

| Organ | Pmax | fTmax | AUEC | fMRT | fT res |
|---|---|---|---|---|---|
| Liver | 1174 | 6 h | 16118 | 12.72 | 48 h |
| Kidneys | 666 | 2 h | 4668 | 10.86 | 48 h |
| Blood | 801 | 18 h | 7743 | 19.89 | 48 h |
| Brain | 58 | 2 h | 291 | 7.63 | 48 h |
| All organs | 1477 | 2 h | 28820 | 14.29 | 48 h |

1. A method for functionally characterising the onset, duration, and resolution of the biological effects of therapeutic or beneficial product, said product preferably comprising one or more natural matrix, following its administration to a subject, the method comprising:
(a) extracting RNA from biological samples of different tissues obtained from living organisms at multiple time points following administration of said natural matrices-based product, hereinafter treated sample(s), and from corresponding biological samples obtained at the same time points from a sham control living organism, hereinafter sham control sample(s);
(b) performing a transcriptomic analysis comprising gene expression profiling on the extracted RNA from each of said treated and sham control samples; and identifying, for each treated sample at each time point, transcriptomic perturbations in terms of significantly Differentially Expressed Genes (DEGs) relative to the corresponding sham-control sample;
(c) defining the onset, duration, and resolution profile of the biological activity induced by the administration of said product
wherein
the onset time point corresponds to the first time point at which a significant transcriptomic perturbation is observed in at least one of said treated samples with respect to the corresponding sham control sample;
the resolution time point corresponds to the last time point at which a significant transcriptomic perturbation is observed in said treated samples with respect to the corresponding sham control samples;
the duration corresponds to the time span between said onset and resolution time point;
the method providing said onset, duration, and resolution profiles of the biological effects of said product independent of direct quantification of individual chemical constituents thereof.

2. The method according to claim 1 further comprising one or more of:
a. providing the product's transcriptomic peak, Pmax, value for one or more organ representing the maximum intensity of the transcriptomic perturbation induced by the administration of said product in said organ,
b. providing the product's functional Tmax, fTmax, value for one or more organ representing the time point at which the maximum transcriptomic perturbation peak induced by the administration of said product in said organ is observed;
c. providing the systemic Area Under the Effect Curve, representing the area under the curve of the overall effect/perturbation induced by said product treatment over time
d. providing the systemic functional Mean Residence Time, fMRT, defining the average effect/perturbation time induced by said product treatment wherein fMRT=AUMC/AUEC, AUMC being the Area Under the first Moment Curve and AUEC being the Area Under the Effect Curve as defined in c
e. providing the functional resolution time, fTres, corresponding to the time point at which the end of transcriptional perturbation, defined as the time point wherein DEGs number is ≤5% of the Pmax for all tissue samples excluded blood, blood end of transcriptional perturbation being defined as the time point wherein DEGs number is ≤12% Pmax, preferably ≤5% Pmax.

3. The method according to claim 1, wherein said different tissues are selected from one or more of blood, liver, kidney, brain, hypothalamus, lung, heart, spleen, testis, stomach, intestine, gallbladder, pancreas, glands, ovaries, muscle tissues, preferably at least blood, liver, kidney, brain, lung, and heart tissues.

4. The method according to claim 1 wherein said multiple time points comprise T0, representing the time immediately after the administration of said product; and at least one, preferably at least two, even more preferably at least three different Tn, wherein n represents the hours after the administration of said product and is ≥1, preferably >2 and wherein one of said Tn is ≥to said resolution time point.

5. The method according to claim 4 wherein said Tn time points comprise T2 and T48, preferably wherein said Tn time points comprise at least three of T2, T6, T18, T24 and T48.

6. The method according to claim 1, wherein said transcriptomic analysis is performed using RNA sequencing or hybridization to a gene expression microarray.

7. The method according to claim 1 wherein said transcriptomic analysis comprises performing a transcriptome raw data analysis from the RNAs extracted in a) and identifying for each treated sample at each time point the significantly differentially expressed genes (DEGs), with respect to its corresponding sham control sample and their expression fold changes with respect to said corresponding sham control samples thereby identifying the number and intensity of the significant transcriptomic perturbations in said treated samples with respect to said corresponding sham control samples at each time point.

8. The method according to claim 1 further comprising extracting RNA from biological samples of different tissues obtained from a control untreated living organism, hereinafter untreated control samples, performing a transcriptomic analysis comprising gene expression profiling on the extracted RNA from each of said untreated control samples and a subsequent Molecular Degree of Perturbation, MDP, analysis in the product-treated corresponding samples and in the sham-treated control corresponding samples thereby obtaining a MDP perturbation score.

9. The method according to claim 8 wherein said MDP perturbation score is calculated according to the following formula:
$${MDP}_{i} = \frac{1}{n} {\sum }_{j = 1} \vert \frac{{x}_{ij} - {μ}_{j} ( ref )}{{σ}_{j}^{( ref )}} \vert$$
wherein
MDPᵢ: perturbation score defining the Molecular degree of perturbation, for subject i
n: Total number of genes used in the analysis
xᵢⱼ: Value of gene j in the subject i
μⱼ(ref): Mean of the gene j in the reference group (untreated controls)
$${σ}_{j}^{( ref )} :$$
Standard deviation of variable j in the reference group
Wherein each gene j is included in the analysis only if its absolute Z-score value is ≥2.

10. The method according to claim 1 wherein said therapeutic or beneficial product comprises or consists of one or more of: cut or pulverized plant parts, plant extracts, fractions of said extracts, microorganisms, honey, propolis, silk, wax, plant resins, plant gums, plant exudates, vegetable oils, vegetable essential oils, animal tissues lysates, or plant or animal fluids, or animal mineral matrices.
